(19) United States
(12) Patent Application Publication
(10) Pub. No.: US 2024/0062878 A1
(43) Pub. Date: Feb. 22, 2024

(54) NEUROPSYCHOPHARMACOLOGICAL TREATMENT EMPLOYING VIRTUAL REALITY

(71) Applicants: Agnieszka Dorota SEKULA, Melbourne (AU); Prashanth PUSPANATHAN, Melbourne (AU)

(72) Inventors: Agnieszka Dorota SEKULA, Melbourne (AU); Prashanth PUSPANATHAN, Melbourne (AU)

(21) Appl. No.: 18/500,921

(22) Filed: Nov. 2, 2023

Related U.S. Application Data
(63) Continuation-in-part of application No. PCT/AU2021/051293, filed on Nov. 3, 2021.

(30) Foreign Application Priority Data
May 4, 2021 (AU) 2021901318
Aug. 20, 2021 (AU) 2021218223

(51) Int. Cl. G16H 20/70; G06F 3/01

(52) U.S. Cl. CPC G16H 20/70; G06F 3/011

(57) ABSTRACT

A method is for treating a condition in a participant, such as a psychiatric condition or another type of personal condition to which the participant is subject. The method includes administering to the participant a therapeutically effective amount of a ΨD and identifying when the participant experiences a peak state evoked by the ΨD. The method further includes providing a VR experience to the participant while experiencing the peak state, whereby a response associated with the peak state is evoked in the participant upon provision of the VR experience when the participant is substantially under no influence of the ΨD.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD

This application claims priority from Australian provisional patent application no. 2021901318 and Australian complete patent application no. 2021218223, the entire contents of which documents are incorporated herein by reference.

The present invention concerns neuropsychopharmacological treatments employing virtual reality, including methods of treating a psychiatric condition in a participant through use of psychedelic drugs and virtual reality.

### BACKGROUND

Psychedelic drugs (ψDs), including plant based ψDs such as psilocybin, have a long history of medicinal use. They have primarily been employed as aids to psychotherapy in the treatment of mood disorders (including depression and PTSD) and substance dependence. ψDs have also found application in treating neurological disorders such as neurocognitive impairment, Alzheimer's disease, autism spectrum disorder and attention-deficit/hyperactivity disorders.

Controlled administering of ψDs can result in an altered state of consciousness (ASC), known as a psychedelic state, often associated with a profound psycho-spiritual experience that can impact the participant's belief system. When an appropriate form of support is offered around those experiences, for example during psychotherapy, psychedelic experiences can become a unique opportunity to examine the mechanisms underlying conscious perception or a tool for facilitating psycho-behavioral change. Studies combining psychotherapy with ψDs have demonstrated positive therapeutic outcomes, including behavioral change (for example, increasing openness to new experiences, creativity or cognitive flexibility) and mental health patients.

The impact of psychedelics can be associated with their neurochemical mechanisms as well as phenomenological features of the peak psychedelic experience. ASCs induced with ψDs include ‘mystical-type’ experiences (MTEs). As will be understood, an MTE has similar characteristics to a mystical experience (ME), and the term ‘MTE’ is used herein to refer to both. MTEs can be induced pharmacologically, neurologically (e.g. by neurostimulation), physically (e.g through shamanic dance), psychologically (e.g. through meditation), or can occur spontaneously. An MTE is characterized by such subjective phenomena as feelings of unity and interconnectedness with all people and things, a sense of sacredness, feelings of joy, peace and awe, a sense of transcending normal time and space, ineffability and an intuitive belief that the experience is a source of objective truth about the nature of reality. As will be understood, an MTE represents a particularly profound phase of ASC, which occurs during the peak psychedelic experience.

Various efforts have been made to objectively characterize MTEs, primarily analysing phenomenological descriptions of MTEs from a variety of participants in order to find a reliable way to identify a subjective experience as mystical. One accepted defining feature of MTEs is a sense of unity (becoming one with all that exists). Other dimensions include: (1) ineffability (ie. that the experience cannot be encapsulated in words); (2) noetic quality (ie. insight into the depths of some fundamental truth or ultimate reality which transcends the discursive intellect); (3) sacredness (i.e., a sense that what is encountered is holy or in some way sacred); (4) positive mood (ie. joy, ecstasy, blessedness, peace, tenderness, gentleness, tranquillity, awe); and (5) transcendence of time and space (ie. conventional experiences of time and space seem to fall away). Additionally, MTEs are associated with paradoxicality, transiency and persisting positive changes in attitudes to self, others and life itself.

These approaches have been used in empirical studies to develop metrics around peak states and MTEs in order to facilitate assessment of mystical or spiritual state other than by reference to religiosity or psychosocial wellbeing. Such mysticism scales include: the Hood Mysticism Scale (M Scale), developed by Hood, 1975; the Mystical Orientation Scale (MOS) developed by Francis and Louden, 2000). Evaluation of MTEs can be conducted using one or more of the known mystical experience questionnaires (MEQs), such as MEQ30, MEQ43 and MEQ6.

Computer-based virtual reality (VR) has also developed in recent times as having the capacity to disrupt the rigidity and limitations of typical conscious experience. Typically, this involves providing specially devised video content, delivered to and viewed by a participant immersively through a head-mounted display, for example providing a realistic 3-dimensional visual scenescape, which may also include auditory content delivered by headphones or other means. In this way, it is possible (for example) to simulate visual hallucinatory experiences. VR can thus be used to alter the sensory experience of users, including evoking particular mental states and emotional responses, including awe. In the clinical setting, VR has been used, often together with traditional therapies, to treat a variety of mood disorders, by transiently altering a participant's perspective and disrupting the rigid patterns of mental experience.

In this specification and claims, virtual reality (VR) includes augmented reality (AR), ie. systems and methods which combine real and computer based imagery, affording enhancement of real world experience with computer-generated perceptual information.

While these two different therapeutic approaches have developed separately in parallel, some thought has recently been given to a composite approach in certain psychological and clinical scenarios, applying both together in order to enhance the effectiveness. For example, it has been suggested that VR might be used to optimize the environment in psychedelic sessions. Further, if the combined approach can be shown to lead to increased efficacy of treatment, a therapeutic effect may be achieved with the use of lower doses of a given psychedelic compound.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY

In one aspect, the present invention provides a method of treating a condition in a participant, including:
administering to the participant a therapeutically effective amount of a ΨD;
identifying when the participant experiences an altered state evoked by the ΨD;
providing a VR experience to the participant while experiencing the altered state, whereby a response associated with the altered state is evoked in the participant upon provision of the VR experience when the participant is substantially under no influence of the ΨD.

The VR experience is preferably a dynamic scenario comprising multiple portions, the method preferably including: when the participant is under the influence of the ΨD, identifying a portion of the VR experience corresponding to the altered state; and, when the participant is substantially under no influence of the ΨD, providing said portion of the VR experience to the participant in order to evoke the response associated with the altered state.

Accordingly, the invention provides a novel anchoring approach, using synergistic properties of VR and ΨD to create anchors of altered state experience for use after the psychedelic effects wane, enabling triggering their recall, easing access to those otherwise ineffable experiences, and therefore maximising the therapeutic use of experiential moments during the psychedelic journey, particularly in integration. The approach of the invention therefore intrinsically links the VR and psychological experience to make use of consciousness triggers in post-dosing therapeutic situations.

Identifying when the participant experiences an altered state evoked by the ΨD may comprise suitable psychological, physiological, behavioral or neurological measures, or a combination of such measures.

Said altered state may be a peak state experienced by the participant (as discussed further below), or may be a determined point in time relative to the peak state. For example, said altered state may be a point in time after the participant's experience of the peak state, such as towards the end of the participant's psychedelic experience.

Further, identifying when the participant experiences an altered state evoked by the ΨD may be performed in real time or at a subsequent time, or by way of a combination of the two.

It should be noted that the phrase “when the participant is substantially under no influence of the ΨD” (when the response associated with the altered state may be evoked in the participant upon provision of the VR experience) envisages the participant being under the influence of a sub-therapeutic dose of ΨD (ie. a mini dose or micro dose, sometimes referred to as psycholytic dose). For example, a sub-therapeutic dose may be equal to or less than: 10 mg (psilocybin); 1 g (dried mushrooms); 10 g (wet mushrooms); 100 μg (LSD); 75 mg (MDMA); 0.2 mg/kg (IV ketamine).

Providing the VR experience to the participant may comprise providing a VR headset to the participant, optionally in combination with one or more ancillary sensory simulation devices. Such a device may be, for example, position-tracking haptic gloves, able to detect motion of a participant's hands and feedback sensory input, eg. when the participant ‘touches’ an object or surface in the VR environment. As will be understood, the VR experience can therefore be designed as interactive or non-interactive (display only).

Providing the VR experience to the participant may include or be combined with provision of an acoustic environment, such as a soundscape and/or music and/or vocal guidance, which may be either pre-recorded or offered in real time by the therapist. The soundscape may be synchronized with the VR dynamic scenario. In accordance with this feature, the method may include providing audio headphones to the participant.

It is recognized that auditory stimuli can significantly influence ‘presence’ or ‘immersion’ in VR, ie. the feeling of ‘being there’ in the virtual environment.

The provision of the VR experience to the participant while experiencing the altered state may be carried out towards the end of the period in which the peak state is experienced.

The method may include providing a different VR experience to the participant as a rescue VR if it is determined the experience has become detrimental. In particular, the rescue VR is different to the portion of the VR experience corresponding to the altered state.

In this way, a rescue scenario may be provided to the participant, the rescue scenario designed to work in opposition to other VR scenarios by pulling the participant out of the experience.

In a further aspect, the invention provides a method of treating a condition in a participant, including providing a VR experience to the participant when substantially under no influence of a ΨD, in order to evoke a response associated with an altered state, said VR experience having been previously provided to the participant while experiencing the altered state under the effects of a ΨD administered to the participant.

Preferably, the VR experience previously provided to the participant comprises multiple portions, and the method includes providing to the participant a portion of the VR experience which has been identified as corresponding to the altered state.

In a further aspect, the present invention concerns a method of treating a condition in a participant using a unified VRPP protocol involving multiple treatment phases, the unified VRPP protocol comprising a plurality of VRPP sub-protocols applied during one or more of the treatment phases, the sub-protocols selected from the group of ASC priming, facilitation, grounding, surrender, restoration, anchoring and rescue, wherein:
at least one of the treatment phases includes the step of administering to the participant a therapeutically effective amount of a ΨD; and
wherein all of the VRPP sub-protocols are unified by providing to the participant under each sub-protocol a VR experience including at least one portion or experiential element common to all the sub-protocols,
so to moderate functions within each treatment phase in a predictable and repeatable way.

Hence, all of the VRPP sub-protocols within the unified VRPP protocol (which could be a subset of the totality of VRPP sub-protocols used in the treatment) are unified by the at least one common portion or experiential element.

The multiple treatment phases preferably include, in addition to one or more dosing sessions in which the therapeutically effective amount of a ΨD is administered to the participant, one or more preparation sessions and one or more integration sessions.

The multiple treatment phases may also include one or more self-practice sessions.

The invention can also be used to augment the emotional experience during the altered state of the participant, the common VR portion or experiential element serving to deepen the participant's emotional reaction when under effects of the ΨD.

Preferably, in either aspect of the invention, the method includes the step of selecting a participant having a condition identified as being potentially treatable by way of the method.

The condition treated by the method of the invention may be a psychiatric condition or may be another type of personal condition to which the participant is subject, such as an interpersonal or intrapersonal crisis or a wish to improve psycho-emotional well-being or general emotional health and overall functioning.

Preferably, the method of the invention is carried out in such a way as to reduce or address the participant's condition. In the case of a psychiatric condition, in a way to ameliorate the condition, otherwise to improve the participant's mental health or benefit the participant's psychological, emotional or spiritual well-being.

In a further form, the invention concerns a method of facilitating the operation of a clinical or therapeutic service or practitioner, including providing guide protocols to the clinical or therapeutic service or practitioner for carrying out either of the above-defined aspects of the invention.

The invention thus concerns a new and effective synergistic coupling between the use of ψDs and VR, arising from the wholly alternative, structured approach taken by the inventors.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 is a flow diagram illustrating an example unified VRPP methodology in accordance with the invention.

FIG. 2 illustrates schematically the process of using VR peak anchors in a psychedelic-assisted psychotherapy (PP) framework.

FIG. 3-5 show graphical representations of the various phases of the intensity of the altered consciousness state of a participant through a VRPP process in accordance with the invention.

FIG. 6 sets out the elements of a dosing phase of a VRPP methodology, illustration the therapeutic interaction and tools applied.

### DETAILED DESCRIPTION

It should be appreciated that all alternative combinations of the features and aspects of the approaches and solutions introduced in this specification and discussed in greater detail below (to the extent that such features and aspects are not mutually inconsistent) are contemplated as being part of the inventive subject matter disclosed herein. It will be understood that the invention disclosed and defined in this specification extends to all alternative combinations of two or more of the individual features mentioned or evident from the text or drawings. All of these different combinations constitute various alternative aspects of the invention.

Further, it should be appreciated that the various concepts introduced herein and discussed in greater detail below may be implemented and applied in any of numerous ways, as the disclosed concepts are not limited to any particular manner of implementation. Examples of specific implementations and applications are provided primarily for illustrative purposes.

The present invention relates to methods and systems for using virtual reality (VR) technology in combination with psychopharmacological compounds (psychedelics) for mental health treatment, psychological well-being and optimal functioning of individuals or groups. As discussed further below, use of the invention can lead to notable improvements in a participant's mental state.

A benefit of using the approach described herein is the provision of a greater and more durable or maintained improvement following treatment with a psychedelic drug (ψDs) capable of eliciting emotional, physiological or psychological effects.

As used herein, the term ‘psychedelic drug’ (or ‘ψD’) includes any compound classified as a hallucinogenic, psychoactive, psychedelic or dissociative drug. Non-limiting examples of such drugs include psilocybin, lysergic acid diethylamide (LSD), ketamine, methylenedioxy-n-methylamphetamine (MDMA), mescaline, ibogaine, ayahuasca or N,N-Dimethyltryptamine (a DMT, such as 5-MeO-DMT). The psychedelic drug can be a tryptamine, a phenethylamine, or a lysergamide.

The psychedelic drug is optionally in a salt form or a solvate form or both. As will be understood, the psychedelic drug may be provided as a pharmaceutical composition including at least one pharmaceutically effective excipient. The psychedelic drug can be an analogue designed to convert upon administration into an active psychedelic drug.

Administration of a ψD is usually oral. For example, it may be a capsule containing between 10 mg and 60 mg (eg. 25 mg) of psilocybin, synthetically manufactured in a laboratory and meeting quality specifications suitable for human research use. The amount of active ingredient will be selected depending on participant body weight, sensitivity, experience, desired outcome and psychotherapy protocols.

In another example, the psychedelic substance used could be a synthetically manufactured capsule containing between 100 μg and 300 μg of LSD, or between 75 mg and 200 mg MDMA.

In yet a further alternative, the psychedelic substance could be a naturally occurring substance, such as between 1 g and 3 g of dried psilocybin containing mushrooms, or a therapeutic dosage of DMT, 5-MeO-DMT, ayahuasca, ibogaine or mescaline.

Administration of ψD can also be intravenous, for example IV administration of 1-2 mg of psilocybin or 0.2-1 mg/kg of ketamine.

As used herein, the term “psychedelic state” refers to the emotional, psychological and physiological state elicited with a psychedelic drug—sometimes referred to as the trip or the psychedelic experience—characterized by unique phenomenological properties attributable to psychedelics and lasting usually between a few minutes up to several hours.

Further, as used herein, the term “psychedelic journey” refers to the totality of the experience around the use of psychedelic substance, from the initial stages of preparation prior to the intended use, through the psychedelic experience and up to and including the stages of integration of the experience following the use of the psychedelic substance, which usually last from days to several months or years.

Further, as used herein, the term “psychedelic effect” refers to the emotional, physiological or psychological influences attributable to the psychedelic substances, including timelessness, ego-dissolution, encounters with ultimate reality or something sacred or divine, connectedness or oneness with all living things, ecstasy or ineffability.

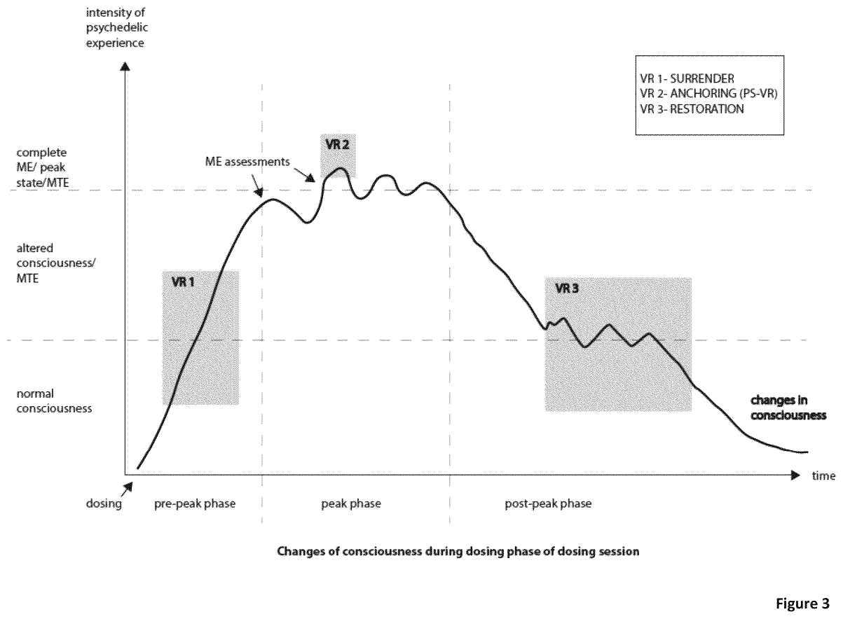

Further, as used herein, the term “peak state” relates to a portion of the participant's altered state of consciousness (ASC) evoked by the ψD, namely the participant's psychological state during a ψD dosing session when the most intense experiences occur, and accordingly the period of time over which the peak state is experienced is referred to as the “peak phase”. For example, with psilocybin there is generally a period 1-3 hours post-dosing in which high intensity states occur, but the intensity of the experience oscillates throughout this time. When it reaches highest intensity this is the peak state. In many cases, the peak state may comprise an MTE, which (as discussed above) represents a particularly profound phase of ASC. This is illustrated in FIG. 3 (discussed further below). Relative to the peak phase are the “pre-peak” and “post-peak” phases of the dosing session, measured by time after the drug is administered. Hence, the pre-peak phase is generally 0-1 hours post dosing and the post-peak phase is generally 3-6 hours post dosing.

As noted above, the term MTE is used herein to apply to situations outside the use of ψD, in particular in religious circles. Current measures often use the term ‘complete mystical experience’ when outcomes reach 60% of the maximum score on the mystical scale used. In the context of the present invention, it will therefore be understood that the peak state may comprise (or include) a complete mystical experience, but this is not essential. Therapy with psychedelics is beneficial, and the advantages of the present invention can be achieved, even if a complete mystical experience is not attained.

The approaches and principles described herein can be applied to many different types of conditions (both psychological and physiological), including but not limited to anxiety, depression, major depressive disorder, obsessive-compulsive disorder (OCD), post-traumatic stress disorder (PTSD), substance-use disorder (SUD), dementia, Parkinson's disease, Huntington's disease or other neurodegenerative condition or Alzheimer's disease.

Indeed, conditions that are potentially treatable by way of the method include all indications and contraindications that are potentially treatable by controlled, therapeutic use of psychedelic drugs, eg. in psychedelic assisted psychotherapy.

Example psychological contradictions include a history of psychosis, family history of psychosis, active suicidal ideation or intent, severe personality disorder, current self-harm and recent history of aggression. Example physiological contraindications include hepatic or renal impairment, cardiovascular compromise and a history of cardiac arrhythmias. Additional contraindications include nausea, sea sickness or epilepsy.

Further, the approaches and principles described herein can be applied in psychotherapy, group therapy, counselling or similar, including with participants without a recognized mental health condition who wish to work on a psychological, emotional or spiritual crisis of some sort.

The approaches and principles described herein can be applied in conjunction with any psychotherapeutic frameworks that are used in therapies using psychedelics, including: cognitive behavioral therapy (CBT); acceptance commitment therapy (ACT); accept-connect-embody model (ACE); accept-connect-embody-restore model (ACE-R); mindfulness therapy and others.

Therapy using ψDs

Psychedelics' unique ability to loosen habitual thinking patterns, increase openness to change, enhance empathy for self and others, and improve neurological and psychological flexibility make them a perfect candidate for conditions, particularly those characterized by rigid, addictive or emotionally overwhelming states of mind. Psychedelic-based treatment represents a unique approach in psychopharmacology due to strong dependency of effects on participant mindset and the environmental setting. An open mind is more prone to heal, but also to absorb all internal and external stimuli. Treatment needs to be carefully designed to promote healthy exploration and reintegration. Psychedelic-based treatments are also referred to herein as psychedelic-assisted psychotherapies (PP).

Traditional psychedelic-retreats followed a nature-immersion approach promoted by early researchers in the field. This context offered a continuous, relaxed, multi-sensory immersion throughout the entire psychedelic journey. Nature-evoked oneness shares similarities with sought after psychedelic effects, including timelessness, pro-social attitudes and spirituality, making it the most successful facilitator of the psychedelic experience thus far.

Clinical models ruptured those unison experiences impeding fluid transition across phases (embodied in the different sessions). They follow the guidelines of the ‘set and setting’ protocol, which divides psychedelic-based treatment into three distinct phases, namely preparation, dosing and integration. Preparation and integration sessions can differ from the psychedelic-enhanced dosing sessions in regard to features of environmental design (e.g. special room set up for dosing session but not used for other sessions), sensory cues (music, nature books, other props during dosing phase only) or therapist involvement (e.g. one therapist instead of the two who were present during dosing), among others. The set and setting approach is not applied as rigorously during preparation and integration sessions as it is during the dosing sessions. The dosing session is detached from the larger context of treatment and has no reflection in its other phases.

It is of value to discuss further the ‘set and setting’ protocol approach here, as this is important to a full understanding of the value of the present invention. ‘Set and setting’ in the context of PP refers to non-pharmacological contextual factors. ‘Set’ refers to the preparation of the participants, their prior psychological traits, personality structure, and their state of mind at the time of the experience. ‘Setting’ refers to the specific physical, social, and cultural environment in which the experience unfolds, and also the broader cultural and media discourse in which the participants are embedded. Set and setting influence the psychological effects of any psychotropic substance (including common drugs like alcohol and nicotine), but it is well understood that PP participants are particularly sensitive to these conditions. Studies show that participants enter into a kind of ‘hypersuggestable state’, in which the impacts of set and setting amplify their susceptibility and responsiveness to suggestions, which have the potential to alter the contents of consciousness, magnifying whatever meaning participants bring to the experience, and significantly influencing their perception, sensation, cognition, emotion, and behavior.

With this in mind, it is not surprising that all phases of treatment impact the course and outcome of ψD psychotherapy. PP study designs show more favorable outcomes in traditional settings as compared with clinical settings, even when methodology is closely matched. Inconsistencies in experience design may be responsible for psychological side effects and waning of positive outcomes with time. Heterogeneity across clinical studies also presents a problem for rigorous, reproducible research.

As mentioned above, therapies which use ψDs commonly involve a three part process: preparation sessions, dosing sessions and integration sessions. Each of the phases of this psychedelic journey can involve one or more sessions. This cycle can be repeated, depending on the number of dosing sessions, each dosing session utilising the same or a different dose to the previous session.

Preparation refers to any therapeutic session that takes place prior to the dosing session. During preparation (which usually involves multiple sessions), the rapport between therapist and participant is established, the participant is introduced to and informed about the way psychedelic experiences works and what can be expected, and the psychological processes and attitudes, which will assist in affording the great benefits from the experience, are initiated.

The dosing session involves the administration of the psychedelic compound. This session generally lasts several hours (eg. 6-12 hours) and is carefully designed and managed around the effects of the drug. After physical and mental preparation of the participant (pre-dosing), the drug is ingested and the therapist assumes a role of facilitator, fully present and available to ensure that the experience is smooth, safe and deeply engaged in, with no more interference with the experience than is essential. The pre-peak phase (also referred to as the come up phase); the peak phase and the post-peak phase (also referred to as the come down phase) are discussed above. A further phase is also often employed, which takes place immediately after the effects are no longer felt or when they have reduced to a residual or minimal level, and involves an initial usually brief narrative or debriefing between participant and therapist. As will be understood, ‘dosing’ refers to the actual ingestion of the ψD, with ‘pre-dosing’ referring to anything that happens during the dosing session preceding ingestion and ‘post-dosing’ referring to the period following dosing up until the end of the dosing session (ie. including the pre-peak, peak and post-peak phases as well as the remainder of the session after psychedelic effects wane.

Integration refers to any therapeutic session that takes place subsequent to the dosing session. Integration sessions generally involve multiple sessions over the course of several weeks which structurally resemble regular therapy. Experiential moments of dosing session are discussed, analysed, evaluated and used in conjunction with other behavioral tools to address the key issues. Structurally, integration can be further divided into debriefing sessions and further integration. Debriefing refers to the sessions that take place closely following the dosing session, usually in the first few days thereafter, though possibly beginning on the day of dosing, and these sessions target the integration of the so-called ‘first narrative’. Further integration usually begins several days after dosing and is repeated regularly for weeks or months.

Use of VR

In accordance with the protocols developed by the inventors, VR is used throughout the psychedelic journey in order to moderate specific functions within each phase. Psychotherapeutic frameworks used with psychedelics are diverse and no unifying structure has been conclusively agreed on by experts. In accordance with the approach of the present invention, VR is used as a central psychological mechanism that unifies all the elements of treatment in a predictable and repeatable way. This is found to be highly beneficial for successful healing, while avoiding the need to determine and adhere to a rigid, concrete psychotherapeutic framework.

As a strong, multisensory catalyst, VR can be used to ensure increased fluidity of the entire psychedelic journey. As will be understood, VR is a ‘full-spectrum’ tool that permits capitalising on exceptional aspects of the psychedelic journey, most uniquely the experiences associated with consciousness expansion, the unique mind-set characteristic of the afterglow phase, and the unexplored opportunity for anchoring peak or altered state experiences. All of these constructs are known for augmenting the efficacy of ψD psychotherapy. In brief, using VR as an adjunct to psychedelic journey can:
Prepare and train an ideal mindset of the participant to encourage a complete surrender to the effects of psychedelics during the dosing session;
Provide an ideal, immersive, safe and comfortable setting during dosing to deepen the absorption into psychedelic effects and minimize side effects;
Improve access, comfort and ease of addressing learning from the dosing session and expand the afterglow mindset to maximize benefits of integration.

VR has a number of benefits over alternative methods. Clinical settings can impair the participant's psychedelic journey and lead to higher levels of anxiety. VR offers a low-cost, straightforward, reliable method for creating a significantly more favorable setting for the participant. While music has been used in psychedelic therapeutic frameworks to distract from the clinical environment and offer sensory cues, VR impacts the sensorimotor system more completely than music and has the capacity to take the patient, experientially, fully out of the clinical setting.

Assessment of psychological interventions shows that methods which require the least amount of skill can be the most accepted and effective for affective disorders. VR does not depend on the participant's ability to imagine or visualize a safe environment or to focus on meditative patterns. Further, VR is reportedly one of the most enjoyable and comfortable forms of therapy, having one of the highest compliance rates and best therapeutic alliance among all forms of treatments, even for individuals unwilling to try any other form of treatment. Recent meta-analysis of 25 meta-reviews has shown that VR therapy leads to substantial, long lasting, real-world generalisable improvements in anxiety-related disorders. The most successful are VR therapies (VRT) that use peaceful, meditative, abstract or nature-based virtual environments.

Within VR, patients are immersed in a safe, soothing environment over which they have control. The sense of control promotes in the participant an open and welcoming attitude to the impending psychological experience.

In addition, VR disrupts the doctor-patient hierarchy, strengthening the sense of equality.

As a co-facilitator, VR can be readily reused in any place and at any time. Hence, integration can (if desired) be continued by the participant at their own pace at home, either between integration sessions (for example during challenging moments when the therapist is not immediately available) or once the integration sessions are completed, in order to maintain the effects of the treatment.

As will be understood, VR does not depend on the participant's ability to imagine or visualise a safe environment or to focus on meditative patterns. Indeed, unlike other approaches, VR offers full replicability and reproducibility of research, allowing for the increasingly more appreciated contextual factors to be shared, explored and utilized by researchers and clinicians worldwide.

VR can be used in each one of the psychedelic therapy phases separately to augment their effects, or be present throughout the entire psychedelic therapy as a unifying factor. It is believed by the inventors of the present invention that any outcome that can be expected to result from any component of psychedelic psychotherapy can be augmented by additional use of VR.

In the preparation sessions, VR preparation scenarios are used to train the participant's psychological flexibility (via training cognitive defusion or deliterisation, acceptance, present moment awareness, etc.), prime occurrences of ASC and relieve anxiety associated with the upcoming dosing session. Each scenario is generally a unique, standalone scenario that targets a unique function, although preferred approaches involve features which unify many or all of the scenarios and maintain continuity across all the phases of treatment. Such common features could be, for example, an introductory scene, a guiding voice or a particular aesthetic used in the VR scenario. These common features form the basis of grounding VR.

During the dosing sessions, VR is used to shape the ideal mindset of the participant via use of the grounding scenario, which is delivered directly prior to dosing. A surrender scenario, delivered at 0-45 minutes post dosing (pre-peak or come up phase), is used to provide an ideal setting once psychedelic effects begin to take place, and guides the participant's emotional experience in the most favorable direction. In particular, the VR is used to enhance the depth of the peak experience. The characteristics of the peak experience depend to a large extent on sensory information and environmental cues, and so VR can be used as the key source of those cues. As discussed further below, during the peak experience, a VR anchoring scenario may be used to form links between intense psychedelic experience and specific VR features, rendering the VR cues as anchors of that state. Later, a restoration scenario delivered at 4-6 hrs post dosing (post-peak or come down phase) eases the return from the intense psychedelic experience back into normal consciousness.

Further, VR can be used to deliver rescue scenarios as needed, ie. in the event the experience becomes detrimental due to intensity, signs of anxiety, or indications of other undesirable psychological or physiological effects. Generally, a rescue scenario works in opposition to other scenarios by providing competing, distracting sensory input to pull the participant out of the experience.

During integration sessions, one or more of the previously applied VR scenarios are used, most commonly the grounding scenario and the anchoring scenario, in order to trigger the same mindset that was present during the psychedelic experience. Facilitation scenarios (surrender and/or restoration) may also be used. Additionally, standalone integration scenarios are used, which can be similar in nature to the VR preparation scenarios but which focus on somewhat different functions (for example exploration of different concepts of self).

Timing

For the preparation and the integration sessions, the number and timing of the sessions and the number of times certain VR scenarios are used are decided in accordance with a variety of factors, including time availability, total session duration, participant-specific issues, progress made, etc. For the dosing sessions, the timing is more specific. The examples below give more information with regard to dosing and VR application schedules trialed by the inventors and found to be effective. In particular, in Annex 1, example timing schedules for a dosing session and an integration session are set out (unified VRPP with psilocybin).

VR Setup

Visual elements are provided to the participant by way of a head mounted VR display, in the form of a 3D computer-generated model/simulation or a prerecorded 180-360 video. In some instances the field of view is fixed while in others head tracking is incorporated to allow for a more realistic visual inspection. The VR equipment may be freestanding, with preloaded content, or may be connected to device such a computer (laptop, ipad, etc.) via a remote connection (Bluetooth, hotspot, etc.) or via a wired connection. Auditory triggers can be provided via headphones, built into the VR equipment or freestanding, via speakers in the rooms, or produced in the environment in real time, for example by way of the therapist speaking or other means (for example, one or more bell). Tactile components can be received either via handheld controllers (for example vibration), physical objects (for example a cube of ice or carved artefact provided by the therapist) or changes in the environment (for example modification of temperature, provision of artificial wind, etc.). Olfactory and palate stimulants can be provided with the use of oils, scents, foods etc.

The participant has freedom to easily leave the VR space if he/she feels the need to do so. The therapist has the ability to switch the VR scenario off if such need occurs.

VR Design

As will be understood, the present invention is not tied to any specific VR scenarios, design elements, technology platform, level of immersivity, or components. Based on testing a variety of different types of commercial products, the inventors have found that certain features of such scenarios are preferred (discuss further below), but there is a large amount of flexibility in settling on specific VR models for use in practising the invention. As will be understood, most important are the targeted functions of the selected VR experience. As will be further understood, the VR system (as well other aspects of the methodology of the invention) may be supported by further sensory inputs, such as music or haptic gloves or other sensory devices, which may or may not be linked or synchronized to the VR scenario.

The VR design is selected to prioritize safety, mitigate overstimulation, and minimize the risk of technical glitches. Whenever possible it is selected to favor a multisensory approach, a key purpose being to increase the number of, and augment the depth of, experiential moments during the entire psychedelic journey.

The approach of the invention is further described below with reference to three possible configurations, which can be seen as parts of a unified protocol within a common functional model.

### VR-Psychedelic Protocols (VRPP)

The approach of the invention is referred to herein as VR modulated Psychedelic-assisted psychotherapy, and the protocols discussed herein are referred to as VR-Psychedelic Protocols (VRPP). These are protocols that utilize VR as the moderator for psychedelic therapy, their key functional mechanisms described below. In particular, VRPP affords reinforcement of the set and setting approach and its expansion to all phases of treatment.

Hence, as used herein, while VR refers to an immersive, virtual scenario that can be administered to a participant via an appropriate headset, VRPP refers to the combined use of ψDs and VR under one or more specific protocols.

As noted above, VR can be used to ensure increased fluidity of the entire psychedelic journey. The goals of the treatment are best achieved by way of a unified protocol which can be seen as three distinct sub-protocols, namely (1) Grounding VRPP, (2) Anchoring VRPP and (3) Facilitating VRPP. These can be used separately or jointly, as each sub-protocol is potentially exclusive from the others. The cohesive nature of this approach unifies the different steps, so promoting a continuous, multisensory experience that becomes a unifying thread throughout the entire psychedelic journey. In particular, by applying the approach of these protocols, the present invention utilizes in a novel way the untapped synergies of VRT and PP.

The table below sets out the different phases and sessions (discussed in further detail below) in an example treatment conducted in accordance with the present invention.

| Session | Phases | | VRPP | Additional VR |
|---|---|---|---|---|
| preparation sessions | collectively, all preparation sessions | | VR Grounding | VR preparation, VR |
| | referred to as ‘preparation phase’ | | | ASC |
| dosing session | pre-dosing phase | dosing ΨD | VR Grounding | |
| | dosing phase | dosing | | |
| | | pre-peak phase | VR Surrender | VR Rescue |
| | | peak phase | VR Anchoring | VR Rescue |
| | | post-peak phase | VR Restoration | VR Rescue |
| | post dosing phase | | | |

| | | | |
|---|---|---|---|
| integration sessions | collectively, all integration sessions | VR Grounding + | VR integration |
| | referred to as ‘integration phase’ | VR Anchoring | |

Using the protocols of the present approach, VR can be used to provide a unique, integrated, multisensory stimulation that unifies the entire psychedelic journey. This is achieved through use (including repeated use) of the most favorable contextual elements, namely elements that are in tune with each phase of treatment, targeting their respective functions over the whole course of treatment. In particular, this approach has the effect of:
i) enhancing and maintaining a hierarchy-free therapeutic alliance that is consistent through all phases of treatment,
ii) mitigating psychological side effects through enhancing the state of relaxation,
iii) helping participants relinquish a need for control by removing familiar cues that keep them tethered to their external world,
iv) encouraging entering the inner world of experience by buffering the external world and inducing the sense of surrender,
v) facilitating release of intense emotions in a safe environment,
vi) providing continuity to the experience of being outside of traditional boundaries of space and time
vii) aiding and easing revisiting of psychedelic experiences after dosing to maximize their therapeutic utility
viii) enabling a smooth transition from preparation to dosing, from dosing to integration and from integration to self-practice with familiar environmental cues.

As discussed below in further detail, anchoring VR, surrender VR, restoration VR and recovery VR scenarios are closely linked with the effects of the psychedelic drug and the way in which those effects change over time, from the moment of ingestion up until the effects disappear. In particular, these effects are synchronized with pre-peak, peak and post-peak phases.

While effects of each ψPD are characterized by pre-peak, peak and post-peak phases, each treatment follows a different timeline, characterized by different durations and different points of transition between phases. Examples below (including the illustrative timing schedules of Annex 1) are based on a time following oral ingestion of psilocybin, with phases occurring roughly at following times post ingestion: pre-peak 0-1 hr; peak 1-3 hr; post-peak 3-6 hr, these times depending on exact dose, set and setting, participant experience and other factors. As will be understood, these phases will differ for other, orally administered ΨDs, for example: LSD (pre-peak 0-2 hr; peak 2-7 hr; post-peak 7-12 hr), MDMA (pre-peak 0-1.5 hr; peak 1.5-4 hr; post-peak 4-5.5 hr), or Ayahuasca (pre-peak 0-1 hr; peak 1-3 hr; post-peak 3-5 hr); as well as for drugs with other administration routes, such as IV psilocybin. As will be appreciated, the above timelines are approximations only; the exact duration of each phase as well as total duration of the experience depend on drug type, exact dose, set and setting, mode of administration, individual participant physiology as well as their previous experience with ΨDs, and numerous other factors.

In accordance with the protocols, the VRT is applied by a therapist, clinician, doctor, nurse, professional medical employer, allied health professional, psychologist, scientist, researcher, or similar. VR can also be applied by a sitter, a community worker, or an entrusted individual. As noted above, in some scenarios, during the preparation and integration sessions, VR can also be applied by the participant on their own.

Grounding VR

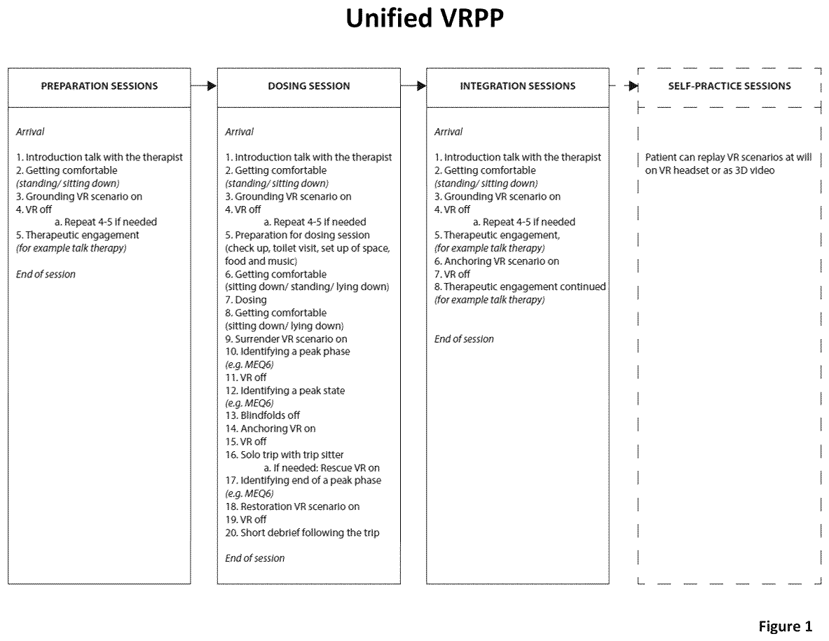

The flow diagram of FIG. 1 provides an example of the unified VRPP approach of the invention, in this case illustrating how ‘Grounding VRPP’ is used throughout a treatment.

As discussed in further detail in Annex 1 below, the grounding VR scenarios are important in maintaining continuity across the entire psychedelic journey.

Grounding VR consists of a single or several VR scenarios that are generally provided throughout all phases of treatment. The inventors have found that the more consistency is kept across sessions, the better the effect: ideally, the exact same or very similar scenario is used each time, during every or nearly every session (for example excluding initial assessment or first introductory session or first narrative session); at the same time in each session (for example, a few minutes after the session begins); and for the same or a similar duration.

Grounding VR scenarios make use of the particular ability of VR to act as a buffering tool, providing isolation from the external world, detachment from familiar temporal and spatial reference points, and hence promoting the state of relaxation and mindful presence needed for the journey ahead. Through the repeated use of grounding VR scenarios this triggering becomes more pronounced and easier with each session.

Grounding VR scenarios can be fully interactive, involving audio, visual and/or haptic components as desired. Alternatively they may be minimally interactive or non-interactive (ie. involving display only).

Such scenarios can take either a directive approach (journey-based, guided meditation, gaming) or non-directive (simply encouraging achieving the desired state of mind and not providing instructions on where to focus or how to do it). For example, a directive approach in meditation scenario is one that uses guidance in form of vocal reminders to remain focused, or challenges the participant to slow down the heart rate with the use of HR biofeedback tool and representative graphics to mark progress. A non-directive approach in meditation might provide a contemplative setting, for example a beautiful landscape, accompanied by peaceful sounds. If grounding VRPP is used in combination with other VRPPs the non-directive approach is preferable, to serve as contrast to the directive approach of facilitation VRPP. Grounding VR scenarios can be fixed (predetermined) or allow high, medium or low flexibility, that is, scenarios can change or adapt in response to participant's decisions. These decisions can be either conscious, through physical or vocal interaction with elements of the scenario and hence affecting where the journey goes or which part of the model is explored; or unconscious, with the use of biofeedback or neurofeedback devices if such are available. Feedback devices can use heart rate, skin conductance, brainwave activity, eye tracking or similar.

Level of stimulation is matched with the desires of the participants; overstimulation should always be avoided and a more relaxed type of scenarios should be encouraged. In inducing the state of relaxation particularly efficacious are self-selected scenarios that represent nature or space.

The therapist may be present in the form of an avatar, visual or vocal representation, and may be present at least in part as a recording. Where group therapy is involved, other members of the group may appear to a particular participant as avatars, visual or vocal representations, and may be present at least in part as recordings.

Scenarios that promote relaxation, mindful presence and buffering often include vast natural or manmade landscapes (a view from a mountain peak, a view over a mountain range, a view over the ocean, a view over an extensive, undetailed man made landscapes, such as a cityscape), nature immersion (a walk through a forest, swimming in an ocean, a walk along a beach, visiting an island), being in space (floating in deep space, for example in near complete darkness or surrounded by stars, a view of planets, galaxies or the sun, falling into a black hole). These examples are characterized by low intensity stimuli, with wide focal points of minimal attention, serving as a contemplative buffer zone to expand awareness. Other designs may involve personally meaningful links to tribal or spiritual practices and places of worship.

While these models are preferred, it will be understood that relaxation and buffering are achieved differently for different individuals, some requiring a stronger stimulus to trigger detachment from daily life. Generally, grounding VR should be individually selected and a full range of features should be made available to choose from, including the type of model, level of interaction, dynamics, focal points, etc. Ideally, grounding VR should avoid overstimulating content, especially content with any particularly negative connotation. A music-based, flow inducing game is a good choice for individuals who require strong distraction.

An example of an interactive, stimulating scenario can include swimming through a deep ocean while performing simple, attention demanding tasks, which may be synchronized with music, for example moving through the ocean at a certain speed in accordance with the rhythm of a musical piece. The scenario can then move onto swimming in the further depths of the ocean where interaction diminishes and action become less dynamic, the participant now surrounded by more spectacular marine creatures, providing a transition into the relaxation phase. Finally, the participant may arrive at the seabed, in the furthest, unexplored depths of the ocean, encouraged by a guided meditation scenario to appreciate the limitless depths of our own consciousness and the unknown nature of what life still has to offer, assisting achievement of mindful presence.

In an alternative scenario, when less stimulation is required, buffering, relaxation and mindful presence can all be achieved via guided meditation scenario and a particular awe-evoking natural landscape, which appears completely natural yet would almost certainly not be attainable by the participant in real life, for example a view from an extremely high peak in an unreachable mountain range.

In most cases, these scenarios will be kept similar across all phases of treatment, in order to offer familiar, reliable cues that can be returned to at any time, also once integration sessions conclude. In some cases, while many elements will remain the same or similar, the scenarios can be slightly modified, for example with changes in length of the immersion, or in certain features of the display, or in the level of interaction. For example, if the VR grounding scenario involves an immersion in a virtual forest environment, either as a 3D model or a 360/180 movie, the same forest scenery can be shown with different daytime or seasonal settings for each session (during sunrise, sunset, midday or night; during winter, summer, spring or autumn), or with different weather conditions (during storm, sunshine, snow, strong wind), etc. Additionally, if certain objects within the environment are movable or interactive (for example, leaves falling from the trees, tree branches moving under wind forces, stones which can be ‘picked up’), changes to those features are allowed. As will be understood, the primary objective is to maintain a perceived continuity, achievable by way of a theme (eg. a forest), a key object (eg. a noticeable large oak tree), or an experience (eg. a walk through a forested landscape).

The grounding VR scenarios can be applied for between 5 minutes and 1 hour, once or several times during the session, in particular at or near the beginning of each session. As FIG. 1 illustrates, grounding VRPP involving these VR scenarios is used in the preparation and integration sessions, as well as during the preparation that immediately precedes dosing.

While grounding scenarios are applied independently of the effects of the drug, other scenarios, which form the part of facilitation VRPP and anchoring VRPP, are linked with the effects of the drug and the way in which those effects change over time, from the moment of ingestion up until the effects disappear. In particular, these effects are synchronized with pre-peak, peak and post-peak phases.

Facilitation VR

As discussed in further detail below, in order for the peak psychedelic experience to be beneficial, the drug-induced transition to and the return transition from the ASC must be mediated by appropriate therapeutic care. Moving the brain out of normal consciousness and into unfamiliar terrain may present some risks if not properly managed. This process is referred to in the context of this disclosure as facilitation VRPP.

With regard to facilitation VRPP, the present invention concerns a method of treating a condition in a participant, including providing to the participant a facilitation VR experience when under the influence of a ΨD but before a peak psychedelic state is evoked by the ΨD, in order to provide a targeted, therapeutic setting via desired sensory stimulation.

In accordance with this approach, the method preferably includes administering to the participant a therapeutically effective amount of a ΨD, providing the or a part of the VR experience to the participant following administration of ΨD but before the peak state is experienced, in order to guide psycho-emotional response associated with the peak state, identifying when the peak state has ended, and providing the or a part of the facilitation VR experience to the participant after the peak state has ended, in order to guide the participant's return to normal consciousness.

In this way, facilitation VR is used to guide the psychedelic experience during the transition from normal to altered consciousness and back again, by working in accordance with the changing effects of the ΨD.

Hence, facilitation VRPP is designed to mediate the experience during the dosing session and provides a bridge between the participant's normal consciousness and psychedelic consciousness (surrender VR) and then back psychedelic consciousness to normal consciousness (restoration VR). Facilitation VR scenarios are the in-between space, which makes this transition smoother, and either deepens the immersion into the experience (surrender VR) or improves consolidation of that experience (restoration VR).

Facilitation scenarios may be selected as very natural scenarios, including those recorded in the real world, for example driving through a forest. Alternatively they may be semi-abstract, for example with familiar references but improbable scenarios like flying through a black hole; or highly abstract, with made-up worlds that are unmatched by the ones found in reality. They can include personally-meaningful concepts or objects, or links to tribal, community or spiritual practices.

Facilitation: Surrender VR

Surrender VR is used during the pre-peak or come up phase (which starts immediately post dosing and ends with reaching the peak phase). For psilocybin this phase is generally the first 60 minutes post dosing. Surrender VR can be applied at any point during this time, but is preferably applied starting around 15 minutes post dosing, with a duration of between 20 and 35 minutes. The therapist ideally ensures a smooth transition from VR to non-VR portions of the session, by preparing the space beforehand, avoiding any unnecessary movement of the participant, helping with mounting and removing the VR equipment and replacing it with blinds and/or headphones as required. Ideally the participant is sitting up in a comfortable way on a bed or couch, which enables the transition to lying down for the remainder of the dosing session.

During the come up phase the physiological response can lead to a sense of tiredness, including a desire to sleep, and yawning is frequent. Pharmacological effects are not yet fully present and cognitive processes, including internal dialogue, remain largely unchanged. This may impair the depth of immersion into the psychedelic experience (through conscious or unconscious distraction) or may negatively impact its quality (e.g. through loss of cognitive focus).

Attention-demanding VR content, designed to maximize immersion and absorption, provides a source of engagement, stimulation and/or excitement, which opposes the tiredness brought on by the effects of the drug. Further, the VR experience occupies the cognitive and emotional resources, distracting from the internal dialogue, which may otherwise either prevent the participant from fully engaging with the experience. Hence surrender scenarios need to be sufficiently engaging to distract from the internal dialogue and sufficiently exciting to mitigate fatigue.

As surrender scenarios require focus, the content is generally dynamic, mildly to strongly stimulating and designed to maintain that focus. Hence, the VR scenario preferably adheres to a number of characteristics, as follows: It is not static, but continues to change regardless of the participant's actions. Ideal scenarios are journey-based, providing a sense of progress with an unknown destination. Movement gives a sense of progress through stages, which may include hints to or glimpses of the destination. Such movement is fluid and not overly radical, although unexpected changes may be introduced. An ideal surrender scenario uses a directive approach, meaning that the story should be experienced as happening to the participant rather than being controlled by him/her. The level of detail is sufficient to attract further investigation for a brief, for example by turning the VR headset to inspect a particular object, without impeding the overall continual progress through the scenario. Overly repetitive elements are avoided. The scenario is presented in the way to minimize resistance and facilitate the flow into the experience.

Multisensory design is encouraged to maximize immersion. Any audio elements are designed to fit the scenario, without necessarily being wholly realistic. For example, floating down the river can be accompanied by an actual recording of flowing water, and/or can also be injected with other, less realistic sounds, alternatively the sound can be completely devoid of realistic references, and (for example) a fitting melody may be used instead. The audio scenario should, though, match the flow and pace of the experience and thus play a role in the absorption into the journey. Other inputs (touch, smell, taste) may be used to play a similar role.

When possible, the multisensory design can be used to link VR portions of the session with non-VR portions. In particular, the audio elements used in the VR may be designed in a way that can be reflected in audio elements delivered via headphones once VR is removed, for example by way of a ‘bridging’ melody. That is, if the VR scenario ends with one melody, the music experience provided immediately picks up from that same tune in order to offer continuity of the experience.

As will be understood, surrender scenarios demand attention, are highly immersive and absorbing, but preferably do not require physical engagement. Any challenge that might be presented to the participant by the surrender scenario should be mental or emotional rather than physical or otherwise interactive. For example the content may be emotionally moving, visuals may be dynamic and bright, demanding a relatively high level of focus that may not be completely smooth or easy. But challenges should demand no more than minimal physical engagement by the participant, if at all. If such minimal physical interaction is included in the scenario this should not be imperative to the progress of the scenario. For example, if the scenario includes the experience of being taken on a boat ride down a river surrounded by trees, and there are leaves falling off those trees, the participant may be able to interact with those leaves physically, ‘catching’ them, ‘releasing’ them, etc., however the ongoing scenario of the boat ride should not be dependent on or dominated in any way by this interaction.

The overall theme is matched with the goal of the psychedelic trip. This means that the participant has a say in determining which scenario is most suitable. While the design itself, its components, its distinct meaning, the destination and sense of movement, etc. should not be revealed to the client, in order to maintain a level of novelty, the theme should be ideally agreed on between the therapist and the client, so to best match the purpose of the psychedelic session. While healing is an overall goal it is achieved via different, more specific pathways and intentions, which are usually determined during preparation sessions and should be augmented during the come up phase of dosing session with the selected surrender scenario. The expectancy effect of psychedelics means that selection of environment will have a strong impact on the psychedelic journey, from spiritual transformation to personal growth.

For example, the goal for having a highly spiritual experience can be augmented with a scenario that portrays driving through a mystical, ancient-looking forest which includes spiritual, mystical or tribal elements, or it could be a scenario involving floating into the heavens. A goal for connectedness might be met with a scenario which is based on a dive deep into the ocean with live creatures joining the participant on this journey, with a progressively deeper connection with the creatures, morphing from physical, to auditory, to energy represented by light, culminating in all of the creatures becoming one with the participant in white noise-filled glow; the need for personal rediscovery may then be represented with an abstract scenario of being reborn.

Themes can focus on references to various forms of self and support formation of that perceived self in the process. For example, if the participant is battling a disease and during preparation sessions a theme of warrior self is identified, the design of surrender scenario should include references to this theme, increasing the sense of self efficacy and strength. This may be represented via the elements of self (eg. holding warrior artefacts), relationship to other creatures (being a leader or commander), or the environment (the participant's movement through the environment breaking physical obstacles in the way).

Surrender scenarios are generally the most personalized of all VR designs. Ideally, these models should be individually designed for each participant. In other embodiments, the basic design (eg. a scenario of movement along a river on a boat) is maintained, while elements of the design are individualized (eg. backdrop, sounds, moveable elements, other forms in that boat, etc.). Alternatively, a number of different scenarios may be available, for selection to match a suitable one to a participant's individual journey.

Surrender scenarios may be applied for 5-60 minutes, ideally for 20-35 minutes, ending around the time of the onset of the peak phase, this phase being reserved for the use of anchoring VR. Following that, in the post-peak or the come down phase a restoration scenario is used. Restoration VR can be applied at any point in this post-peak phase, preferably for a duration of 20-60 minutes. If requested by the participant and it is found conducive to the experience or therapeutic effects, the final scenes of the restoration VR may continue for as long as the participant or therapist sees fit. The therapist should ensure a smooth transition from non-VR portions of the session into VR portions, by avoiding any unnecessary movement of the participant, helping with removing blinds and/or headphones and then mounting and removing the VR equipment. Ideally, the lying position (usually assumed during peak phase), can be preserved for the restoration VR. The therapist can thus replaces eye blinds with VR equipment without any additional changes to the set up between peak and post-peak phases.

Anchoring VR

As noted above, the emotional, psychological and physiological state elicited by the psychedelic drug is referred to as an “altered state of consciousness” (ASC). This altered state involves a noticeable (often dramatic) and qualitative alteration to the participant's fundamental subjective ‘fabric of awareness’, typically accompanied by alterations to perception, cognition, thinking and behavior. In other words, the altered state involves a profound disruption or deviation from regular cognitive and emotional functioning (ie. the baseline psychic functioning) that dramatically transforms the phenomenology and architecture of the subjective experience of reality and selfhood.

During the altered state, and in particular during the peak phase, participants are transported to an alternate reality, where contact with their everyday environment is severed. It is a profoundly altered state of consciousness, during which the intensity of psychedelic effects is the highest, and the peak experiences occur. Closed-eye visual hallucinations are common. These experiences impart to the participants substantial personal meaning and spiritual significance and are often characterized as among the most meaningful of a lifetime.

It is the ability of psychedelics to disrupt stereotyped patterns of thought and behavior by disintegrating the patterns of activity upon which rest accounts for their therapeutic potential. Cognition becomes less constrained and neurodynamics less ordered. By breaking down the stable spatiotemporal patterns of brain activity psychedelics have the ability to dismantle reinforced patterns of negative thought and behavior, while providing novel, spontaneous insights about “self” or “nature”. The lowering of psychological defences allow personal conflicts to come to the fore that can then be worked through with a therapist; while the relinquishment of “ego” enables a more relaxed, flexible and open manner of approaching those conflicts. Hence, peak effects allow the state in which the mind can reshape itself, the process often associated with ego-dissolution or “disintegration”. Magical thinking may occur, characterized by cognition in which supernatural interpretations of phenomena are made.

As noted elsewhere in this disclosure, VR scenarios can be used to augment the intensity of altered state and peak experiences, using the ability of VR to evoke a sensation of awe. VR and ΨDs can thus work in synergy, the ΨD preparing the mind to absorb the stimulus in an unquestionable manner by lowering reality testing mechanisms, the VR providing the intense, vast, unknown and stunning stimulus and thus increasing the intensity of the peak psychedelic state.

The stimulus provided during the altered state of the participant is therefore of high importance, including the stimulus provided during and relative to the peak state. The mind is very susceptible, suggestible and responsive. The peak experience and the experiences around that can be of celebratory, melancholic, terrifying, overwhelming, exhausting, passionate, vital or loving nature. While augmenting the overall sensation VR should preferably be designed to avoid determining the content of the insights. The stimulus should ideally be positive and aesthetically pleasing, aim to inspire, encourage further exploration, set the positive mood, release intense emotions, including the sensation of awe and aesthetic arrest.

Anchoring VR should preferably stay clear from familiar elements; the content being abstract and distinguishable from real life objects. The experience can thus grab the participant's attention, being engaging, entertaining, beautiful and preferably unprecedented. Such novelty helps trigger unfamiliar sensations, plays a crucial role in evoking awe, and allows the creation of the VR anchors (described below). Any scenario personalisation, which requires participant choice, should preferably be avoided.

Design of focal points should satisfy both the sense of vastness and the aesthetic appreciation. The first is most often accomplished with vast landscapes, ideally with vistas that are not readily available in the real world—for example from the highest mountain peak. The latter is best accomplished via aesthetically stunning objects that are human scale; either natural or manmade. These can include stunning, exotic flowers or intricate architectural engineering. Aesthetic arrest is a form of profound aesthetic appreciation accompanied by the sense of understanding of the design—either by nature or by man. These detailed VR elements, while intuitively recognisable, have no real relation to familiar objects; for example organic shapes of intense colors, layers and lightness that do not exist in real life will be intuitively recognized as flowers. This dramatic change in scale corresponds to vastness and accommodation of the awe mechanism.

The anchoring VR may be applied for between 5 and 20 minutes during the peak state. Content is relatively short but dynamic, and there is no need for the scenario to present a coherent story. Stimulus should be powerful, fully immersive and absorbing, completely taking over participant's thoughts and emotions, leaving little space for distraction. Ideally, the scenario involves a 360 view that can be surveyed, with happening in a uniform direction. The anchoring VR used may be the most video-like of all the VR scenarios employed, with little or no interaction and changes happening to the participant rather than with any active engagement. Any movement through the scenario occurs naturally without the need for any physical interaction. Ideally, no handheld controllers are involved, the experience should be effortless.

While the anchoring VR can be the VR applied at any suitable stage while the participant is experiencing the altered state of consciousness (ie. during the peak phase), research has suggested that the ideal timing may be during the later portion of the altered state, ie. towards the end of the altered state phase or when a substantial portion of the altered state has concluded. This can help reduce the likelihood of the VR itself contributing to or otherwise potentially interfering with the remaining part of the altered state experience.

In this regard, the mechanism of anchoring VR works well not just for the peak state of the psychedelic experience but for any pivotal, deeply emotional or altered state experienced by the participant. Hence, the anchoring VR can be the VR applied at a point in time after the participant's experience of the peak state, including at or towards the end of the altered state (ie. towards the end of the participant's psychedelic experience). Research has suggested that the insights generated during the peak state are best recorded at a time after the peak state has concluded, as anchoring can work better if the patient generates unique anchors of the experience himself, as opposed to the provision of abstract anchors. Accordingly, it may be beneficial if the participant is able to interact in a meaningful way with the model, and this can be more complicated during the peak state. The timing of the anchoring VR will depend on a number of factors, including the particular psychedelic employed. For example, the altered state invoked by ketamine has relatively short tail period, meaning that the anchoring VR may be that identified as the VR applied very shortly after the peak state (but not during that peak state). Thus, the point in time of the anchoring VR may be at a selected time after the participant experiences the peak state.

In this regard, the discussion below relates to the anchoring VR as being the VR applied during the peak phase, but it will be understood that this is only by way of example, and the anchoring VR may be the VR applied at other times during the participant's altered state of consciousness.

VR elements should be highly engaging and continue to change, but kept within a certain theme. If the environment changes the number of changes should be limited, or the theme should be kept consistent throughout. This ensures that creating VR anchors is specific and targeted, the trigger concentrated into a relatively specific, novel stimulation.

Anchoring VR is preferably the most dependent on the multisensory character of the VR setup of all the VR scenarios used. Small scale VR elements should be well detailed, with shadows and movement revealing their weight, texture, temperature. Visuals should be comprehensive enough to trigger other senses. Soundscapes used with the anchoring scenario should be abstract and non-melodic. Sounds may be regular, cyclical, generally homogenous. Other senses can be engaged in a way that corresponds with and enhances the 3D content. For example, temperature can be modified with an electric blanket; tactile sensations can be provided with objects that match those viewed in the VR (for example, a soft fan can represent a wind presented in the VR scenario); smells and taste can be involved. The more immersive the experience the more intense it will become. The more senses are engaged in a uniform fashion the more powerful the capacity for the setup to become an anchor for future integration sessions. All of these sensations should preferably be as novel as possible. Music used should preferably not be a continuation of the pre-peak music but rather offer a marked auditory difference.

As discussed below, the anchoring VR applied during the peak phase of the dosing session is then used when the participant is not under the influence of the ψD. Each use of anchoring should be the same, ideally using both the VR and the non-VR elements in a way that replicates that first application as closely as possible.

An example anchoring VR scenario begins in a vast landscape, a desert, surrounded in a distance by beautiful mountains and spectacular sky. Instrumental, non-melodic music is emotional and heavy but not fast. In the middle of the vast desert a tower reaches all the way to the sky. The participant moves towards it, approaching it in a straight line while able to look around at the sky, at the mountains, without affecting the movement. The tower is man-made, monumental, beautiful, light, simple. The tower is filled with multiple, clean cut, arched openings; the staircase that leads up to the top is partially inside the tower and partially outside. The participant enters the tower and the movement upwards begins, smoothly, uninterrupted. The scenario takes the participant up the stairs, towards the top of the tower, moving either inside of the manmade structure or outside, looking out on the vast landscape. The inside of the tower has a spiritual character, it is filled with light, with beautiful architectural elements, human scale structures made of steel, timber or glass, with several lighting sources. The space is habitable but not inhabited. Structures resemble basic architectural elements, provide a sense of human scale and allow admiration at human-created objects; but they are not associated with any specific functions that are associated with familiar activities. They represent architectural sculpture, intricate, requiring impeccable crafting skills. The higher up the participant gets, the more abstract and colorful the inside of the tower becomes and the shapes more organic, flexible and soft. The top is immersed in the lightness of the sky and as it is reached a soft white glow embraces the participant, ending the scenario by floating in this endless, warm, embracing light with white noise sounds.

During the post-peak phase of the experience pharmacological effects of the drug begin to wear off the psychedelic effects slowly weaken. A normal wakeful consciousness begins to return. This can be either a slow or rapid process of return to usual cognitive processes. Given the unique nature of the psychedelic experience, which is characterized by a sensation that what has been experienced was the ultimate reality (or a reality more real than that familiar to the participant), to some this may seem as the opposite of a welcome return and instead be felt as a removal from a profound experience into a perceptual hallucination or a sensory deceit or artificial construct. In other words, there can be a marked change between one state and another and to some this may seem like two opposite forms of consciousness.

The more powerful the ASC experience, the more critical and valuable it can be, but also the more substantial and confronting can be the sensation of return. This is therefore period of vulnerability that needs to be supported. Feelings that come up require a good space for judgment-free reassurance, which can be greatly facilitated with VR.

At the same time this is a very potent period, as the participant has access to both the ASC (though not peak) experience and cognitive processes that are able to make sense of it, either simultaneously or oscillating between the two. This capacity should preferably be enhanced and utilized to create a first narrative of the experience, that can be then processed further during integration. It is important that the participant stays fully present and rooted in the remaining phenomenological sensations brought on by an altered consciousness.

The “first narrative” refers to the process of communicating the experience immediately after it occurs, either at the very end of the dosing phase or shortly thereafter. It is recognized as a beneficial process that can be invaluable for further integration. One of the important and difficult aspects of the psychedelic experience is its ineffability, ie. the sense that the experience cannot be described adequately with words or that words cannot do it justice. This poses a challenge to talk therapy. First narrative can greatly help with this challenge; it is expressive, often somewhat incohesive and requiring later analysis; it is a free flow recounting of the experience and does not need to make sense at the time.

To accept the validity of the experience it is important that the psychedelic experience is viewed as the participant's own, with an internal and not external source. VR can be used to provide a point of difference or reference: it is an externally induced alteration of perception, contrasting with the internally evoked, and therefore more personal and genuine psychedelic experience.

Facilitation: Restoration VR

The primary goal of restoration VR is thus to ease the transition to normal consciousness and maximize the benefits of this ‘in-between’ conscious state. Restoration VR can thus be used to blur the clear distinction between the psychedelic experience and wakeful consciousness and smoothly shift one consciousness to the other, creating awareness that the two are not exclusive of one another, that the expanded psycho-emotional capacity that was reached with the help of the drug occupies the more extreme ends of that very same spectrum that the wakeful consciousness occupies. Thus, the learnings, realisations, ideas and thoughts that occurred with the help of psychedelics can be carried on into the real world.

Restoration VR is applied for the duration of around 20-35 minutes, the duration being dependent on participant's choice. As will be understood, it is applied after the peak phase has ended and when the participant begins to show signs of normal consciousness returning, by for example becoming talkative, beginning to open the eyes or fidget. It can also be self-determined by the participant acknowledging and declaring their return to an ordinary state of consciousness. For example, for psilocybin, restoration VR is ideally applied around 30-60 minutes after conclusion of the peak state.

While restoration VR scenario is generally not static, changes are used to symbolize the return from the journey, and are thus used differently to surrender VR. Generally, changes occur more slowly and are less marked, allowing the participant time to ponder the experience, to contemplate specific components or simply to immerse in the overall feeling produced. The aim of the dynamic is therefore to represent personal transformation, for example instead of moving through the scenery, the scenery surrounding the participant might transform around them.

Aspects relevant to the first narrative can be reflected in the restoration VR used, with abstract, multisensory representations that encourage further exploration. The ineffable experience that is so difficult to communicate during face to face talk therapy can be represented in an abstract way or in a manner that is not wholly coherent. The role of VR at this stage is to provide a transition space which is safe and not yet bound to restrictions of reality, is non-judgemental, flexible and free, in which thoughts can be shared in no particular order. The role of the therapist during this phase is to collect those accounts as they come, encouraging this flow of recounting experiences without obstructing it or trying to contemporaneously make sense of them. For example, encouraging the participant to continue describing the experience or their thoughts is beneficial, but understanding what it means or how it relates to real life problems or challenges can impede this flow and are generally to be avoided at this stage.

As with other scenarios, multisensory VR design is encouraged. During restoration, tactile sensations can be particularly important, as in this part of the session uneasy physical sensations may occur. Pain, tension, anxiety or distress following the experience are not uncommon and physical presence can offer reassurance. If such distress is present, VR which uses focused body work, or offers elements of focused physical work, can be beneficial. Bringing the participant's focus back to their breath, using VR-based breathing exercises, can also offer a tactile reminder of the link to the body.

The therapist can be present within the restoration VR, for example in the form of an avatar, an abstract visual representation, or simply by vocal communication with the participant. Vocal communication may be tolerated better if the therapist uses a microphone and the participant received the audio through the VR headset or headphones or speakers, to avoid creating the distance between the participant who is immersed in the VR world and the therapist who is present in the real world. An abstract visual representation of the therapist within the VR model may be, for example, as particles of light harmoniously incorporated into the model.

Multisensory restoration VR design can be benefial. During restoration, tactile sensations can be particularly important, as in this part of the session uneasy physical sensations may occur. Pain, tension, anxiety or distress following the experience are not uncommon and physical presence can offer reassurance. VR which uses focused body work, or offers elements of focused physical work, can ease such distress if present. Bringing the participant's focus back to their breath, using VR-based breathing exercises, can also offer a tactile reminder of the link to the body.

Restoration scenarios are generally the VR models that benefit most from personal control and responsiveness of the model. Ideally, however, this control should be achieved without the use of hand held controllers, but rather with less demanding methods, as the mind and body are still recovering from the experience and the body should remain relaxed. Forms of interaction other than hand controllers can be employed, and biofeedback devices can be utilized (such as heart rate monitoring, pulsometer, neurofeedback connected to the VR headset, eye tracking system, etc). Alternatively or in addition, changes in the VR environment can be prompted in response to the participant's voice, either directly through a vocally responsive VR design, or indirectly, by vocalising changes in sensations or emotions that the participant is feeling and the therapist introducing those changes to the VR model in real time. These changes may represent the individual mood or state of mind of the participant, or the pace of change at which they return to wakeful consciousness. For example, if the scenario involves a model of space, the brightness, proximity or shade of stars can be adjusted depending on verbal feedback. If a thought or realisation (not necessarily fully formed, but an abstract idea) that arose from the psychedelic experience is vocalized by the participant, a star can shine brighter (representing that single realisation) and move towards the participant, the light becoming one with him/her, so that the realisation becomes symbolically internalized. If no feedback or personalisation is uses, such changes can occur at random, but will not be as aligned with specific thoughts or ideas.

Ideally, a restoration VR scenario starts in complete darkness, to minimize the transition from blindfold to the VR headset. Guidance should be minimal and only provoke certain sensations, allowing enough time for the participant to dwell on their experience. Encouragement can be offered to share thoughts on the experience, suggesting single word associations or key realisations to be shared in the safe environment. Ideally, the therapist uses a microphone to communicate with the client and encourage the first narrative. Real time communication is preferred over pre-recorded guidance that could be distracting.

A sample of a restoration VR scenario is as follows. After an initial few minutes of blackness, very dim lights begin to appear. Slowly, one by one, some of the lights begin to grow bigger and glow brighter. The landscape begins to resemble a model of space and lights begin to resemble stars. As discussed above, if feedback is used at the time, individual lights can be used to link in with thoughts, ideas, realisations and memories from the experience. Each idea, once expressed, attracts a specific light closer, which becomes brighter and stronger, appearing to come closer and closer to the participant until it disappears from view as if it were inhaled into the body. Simultaneously, a sound resembling a deep inhale is provided, the entire VR space expanding and contracting to invite participation in this process of inhaling and exhaling, of embodying the light just inhaled. At the same time, brief guidance can be offered on breathwork. The participant is invited to embody the sensation of inhaling the light and to share any immediate thoughts or sensations that come to mind in association with the experience. Each ‘star’ can guide a different realisation, this process repeating several times, with various tones, lights and configurations, over several minutes of the experience. The lights then begin to float and very slowly the abstract scenery changes, working its way back from abstract mystical shapes that slowly begin to resemble more familiar natural landscapes, a colorful sky that resembles aurora borealis becomes more and more settled and familiar; the light sources that were previously stars begin to fall, initially appearing as shooting stars, slowly increasing in number and transforming into droplets that turn into an all-encapsulating tropical rain; the ground beneath, initially vast and empty, begins to take shape, become solid and reassuring. The scenario ends in a beautiful, natural looking forest immersed in sunshine.

The last scene from the restoration VR can be used as a meditation background to conclude the dosing session.

Referring once again to the anchoring of peak experience (see above), it is also possible—to some extent—to anchor post-peak experiences. This can be achieved by using the restoration VR also in the integration process. The goal of this aspect of anchoring is more cognitive than emotional, when compared to the use of peak anchors. During the dosing session, restoration VR can aid in solidifying memories of the peak experience and, when the same VR is later used during integration, those memories can be revoked, reminded and accessed by way of encoded VR symbolism. This mechanism is thought to work as a memory retrieval process that assists the participant to remember insights that formed during the first narrative process, allowing the participant to delve deeper into those insights during integration sessions.

Additionally, a different type of VR scenario can be used in the case of emergency, referred to herein as rescue VR. If psycho-emotional crisis emerges, and cannot be otherwise resolved (for example by encouraging the participant to work through it), rescue VR can provide a safe, quick, easy and well-tolerated method for suspending the effects of the ψD.

Rescue VR acts in opposition to the effects of the ψD; it is interactive, requires physical action, engagement with the virtual world, and its content is preferably non-dramatic and non-spiritual. VR world is entertainment-based, music is recognisable and melodic, content is dependent on participant's actions. An example is a musical game, in which the participant is required to interact with VR elements in response to a well-known song. Rescue VR can be applied immediately it is required and maintained for as long as necessary, but the therapist should encourage return to the experience as soon as possible, while offering further reassurance and support if needed.

Peak Psychedelic state

As discussed above, peak psychedelic state occurs at different times for different ψDs. There is usually a period of time during which peak state may occur, and the intensity of the experience can fluctuate during that period (illustrated in FIG. 3).

As will be understood, identifying the peak state during the dosing session is key to the present invention. While identifying the peak state is not necessarily important from a clinical standpoint for all types of PP, various methodologies have been developed in the past and will be understood by the skilled reader. These may involve psychological, physiological, behavioral or neurological measures (or a combination of such measures), and may be based on observational or measured behavior (or a combination of the two). Some examples of suitable approaches are discussed below.

Physiological markers may use neural correlates based, for example, on EEG records assessed by an experienced clinician. These may include changes in the power of frontal and temporal theta oscillations. Other physiological markers of the peak state may include heart rate, skin conductance, brainwave activity, eye tracking, eye pupil size or similar. As an example, with the use of functional MRI, decreased functional activity of the brain has been localized in high-level association cortices (including key regions of the default mode network and task-positive network). In another example, alpha power has been found to decrease in the posterior cingulate cortex. These decreases correlate with subjective measures of phenomenological experience of ego dissolution during an ASC.

Alternatively or in addition, mystical experience behavioral observation by experienced therapists may be employed. Further, direct participant feedback can also be used, for example a response to the question “has the intensity of the experience increased in the past 15 minutes?”. If response to the question is “no” more than twice an assumption can be made that the peak experience has been reached.

Other subjective responses can be used, based on the therapist's experience with psychedelic drugs and/or with MTEs, including use of suitable equipment. For example, the participant may push a button or provide a vocal annunciation to indicate attainment of peak state. The participant's experience can also be rated in accordance with a recognized phenomenological scale such as Hood Mysticism Scale (M Scale), Mystical Orientation Scale (MOS), Hallucinogen Rating Scale; Altered State of Consciousness Scale (eg. 5D-ASC or OAV), or results of a Mystical Experience Questionnaire (MEQ) or a States of Consciousness Questionnaire (SOCQ). Ideally, the assessment(s) selected this should be brief so as not to disturb the participant's experience. For instance, a shortened version of an MEQ with only 6 questions may be used.

Other methods of peak state assessment that may be used include multi-modal bio sensing, which combines the use of measures selected from high density EEG, electro dermal activity, heart rate, heart rate variability, analysis of facial expressions, analysis of body movements, conducting experience sampling either through vocalizations or using joysticks or other feedback mechanisms.

Ingestion of psychedelics is known to effect the level of certain chemicals in the body. For example, the hormone oxytocin is measurable, its level (pg/mL) rising under the effect of one or more of LSD, MDMA and d-amphetamine. Hence, as will be understood in the context of this disclosure, identifying when the progress of psychelic experience, including when a participant experiences a peak psychedelic state evoked by a ΨD, can correlate with levels of such chemicals.

VR Peak Anchors

As will be understood from the above and the example protocols set out below, a particular facet of the approach of the inventors concerns anchoring VR, being the VR administered when peak effects are reached during dosing (usually around 1-4 hours post dosing, preferably in the later part of this phase, as discussed above), to provide a way of linking to the peak state. This may be a relatively short, targeted application of a particular multisensory VR stimulation, selectively applied when the participant's emotions reach the highest intensity. Alternatively it may be a particular section of the VR scenario that correlates with the achievement of peak state. In this way, ‘peak anchors’ in the VR scenario are identified.

VR peak anchors are achieved with scenarios that employ specific, unfamiliar, non real-world elements that are conducive to emotional release.

As will be understood, the anchoring VR may be used not only to provide the peak anchors but also to augment the emotional experience during the peak state, which can help maximize the therapeutic use of experiential moments during subsequent integration. VR can be used to apply stimuli that are unprecedented and unexpected. When applied during the peak experience, the attention demanded by such stimuli can renew focus, awareness and/or sense of agency over the experience, injecting new vigor and enthusiasm to afford continued further exploration of that experience. The VR can also be used to deepen the sense of surrender to this part of the journey. Further, it can assist in relieving any fatigue or sleepiness in the participant that may be caused by the psychedelic.

This highly synergistic use of multisensory, highly absorbing VR stimuli, along with the peak psychedelic effects can give rise in the participant augmentation of emotions and sensations in unparalleled ways, producing states of awe. VR augments perceptual, psychological and emotional sensations brought on by psychedelics, deepening the ability to engage with states of gratitude, connectedness, self-realisation, mindfulness, love or forgiveness; as well as surrender to challenging emotions including pain, vulnerability, inadequacy, irrelevance or loneliness. In parallel, psychedelics increase absorption into the VR world, and hence the sense of immersion and relatedness to VR scenarios that produce amazement, aesthetic arrest, joy, admiration or wonderment.

The inventors have realized that, through jointly producing such profound, unprecedented states, VR and psychedelic experiences become intrinsically linked. A targeted, multisensory VR stimulus becomes an anchor to evoke those peak sensations, emotions and mind states, attributing the VR scenario with the capacity to evoke them during subsequent non-dosed sessions.

This ‘peak anchor’ approach therefore identifies a specific multisensory set of cues for the participant which, by being applied during a highly potent emotional state, become mentally attached to that emotional state. Those peak anchors can then be used to enable evoking that same emotional state at will, including when the participant is in a state of ordinary consciousness, by application of that same or very similar VR. In this way, the therapeutic treatment affords access to psychedelic-evoked emotions and cognitions, absent other effects of the psychedelic substance, most notably the inability (under the influence of the ψD) of the participant to communicate and articulate in a cohesive manner. This gives significantly enhanced ability to integrate these effects, giving therapists and participants more time and cognitive resources to beneficially work through stimulating, unexpected or challenging emotions that arise in the process.

This VR peak anchor approach allows prompting the participant's memory of the peak state with multisensory anchors rather than conventional verbal clues, and recalling those emotions will therefore meet less resistance from the conscious mind and become more exploratory, allowing access to deeper, less explicit layers of the participant's experience. In accordance with the invention, then, VR is used as an anchoring catalyst to facilitate ineffable internal recall, symbiotic with unique characteristics of the psychedelic state.

As a multisensory medium, with properties that in many ways can be synergistic to psychedelic states, VR offers novel and intense stimuli that can be reapplied multiple times in a precisely the same manner. This robust repeatability means that VR is an ideal tool to anchor the source emotional states that accompany the peak psychedelic experience and subsequently release it at will, in order to aid the integration process. This can be used to maximize therapeutic use of experiential moments during integration, enabling the translation of powerful but ineffable states into real world outcomes.

As discussed above, following the dosing session, VR anchors play a role in facilitating integration of psychedelic experiences. Anchoring improves access to experiences that are impossible or difficult to communicate while under the influence of the drug. A key benefit identified by the inventors is the ability to expand the sensation of afterglow. Repeated use of peak anchors that hold strong emotional links to peak experience maintain the unique psychological and emotional flexibility, openness and positive mood that are characteristic of afterglow, and this can extend the time available for therapeutic engagement when it has the highest potential to bring beneficial outcomes.

Anchoring VR scenarios should preferably be minimally interactive or non-interactive. The anchoring VR is applied for between 5 and 30 mins, once or several times over that period. As described above, the same or a very similar anchoring VR scenario is then used during integration sessions, both in the initial debrief sessions and during further integration sessions. To maximize its potential, the first few debrief sessions that use anchoring VR scenarios should follow the dosing session relatively soon, preferably as often as feasible in the first 2-3 weeks post dosing (for example, every other day). Anchoring VR scenarios are used in a similar way during further integration sessions. In addition, if possible, the anchoring VR scenarios may be provided for use by the participant at home in a self-practice protocol.

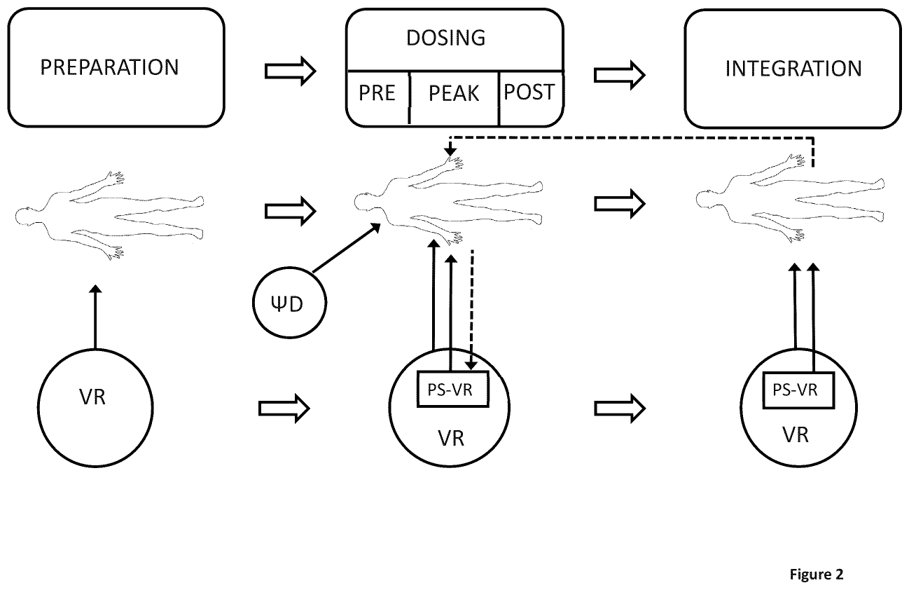

FIG. 2 illustrates schematically the process of using peak anchors in a PP framework. In particular, this figure shows the unfolding of the process with reference to the different treatment phases and with reference to the application of VR. As discussed above, VR is an inherent component of at least a part of each phase, while the anchoring VR (referenced as ‘PS-VR’) in FIG. 2 is the VR applied during the peak state part of the dosing session. The dotted arrow represents the identification of the PS-VR by way of identification of the peak state in the participant. As the right hand portion of FIG. 2 shows, it is this PS-VR that is applied as a peak anchor during the subsequent integration session. The dotted arrow represents the emotional and psychological link to the peak experience achieved by the participant during the dosing session.

FIG. 3 provides a graphical depiction of the pre-peak, peak and post-peak phases of a dosing session, the vertical axis representing the intensity of the participant's psychedelic experience, in other words a measure of departure from normal consciousness. This illustration of the process of transcendence is generally supported by studies into neural correlates of the psychedelic state, including brain imaging data, which show changes in dissociation patterns (reduced activity in the parts of the brain responsible for conscious control). Such data includes the results of neuroimaging carried out using functional magnetic resonance imaging (fMRI), generally involving IV infusion of psilocybin instead of oral administration (as this leads to significantly more rapid changes), and these studies have contributed new insights into the neural underpinnings of qualitative conscious experiences, including MTEs.

In this regard, reference is made to research by Carhart-Harris R L et al, showing that changes in connectivity and activity in particular brain regions is inversely correlated with phenomenological sensation of transcending normal consciousness. The researchers found that psilocybin effects included decreased blood flow in the thalamus, anterior and posterior cingulate cortex, and medial prefrontal cortex, these areas of the brain being key connector hubs, which when faced with a decreased activity, enable a state of unconstrained cognition. Further, Majić, Schmidt and Gallinat “Peak experiences and the afterglow phenomenon: When and how do therapeutic effects of hallucinogens depend on psychedelic experiences?” Journal of Psychopharmacology, February 2015 discusses various approaches in clinical applications of psychedelics, and includes representations phenomenological changes in consciousness induced by psychedelics, similar to FIG. 3.

Particular portions of the graph line in FIG. 3 depict MEs (and MTEs) which may or may not be present at the more extreme phases of ASCs. In particular, the peak phase is represented by the undulating plateau of the line, and it is in this phase that the anchoring VR, VR2 (PS-VR) is administered. VR 1 depicts surrender VR, while VR3 depicts restoration VR (facilitation VRPP being represented by VR1+VR3).

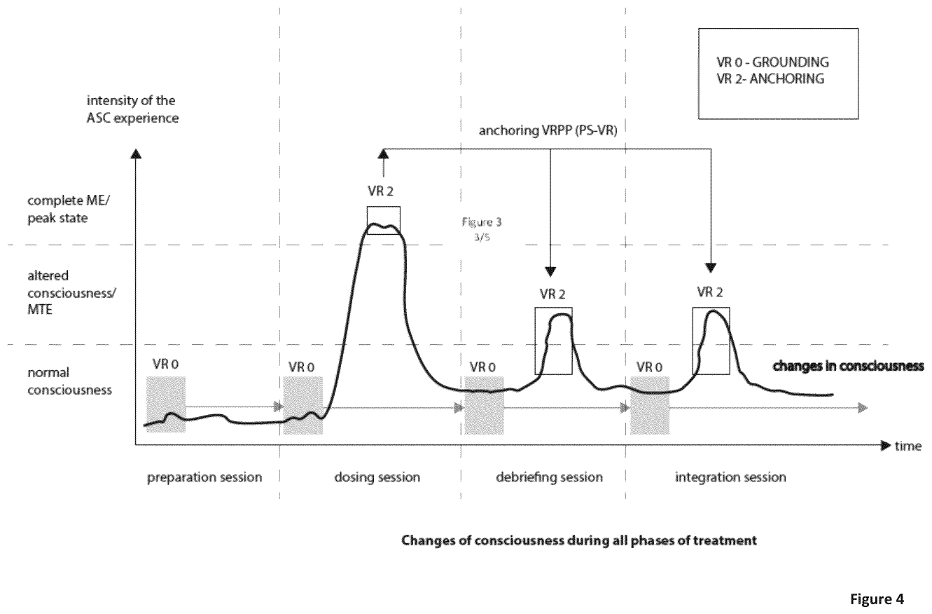

FIG. 4 provides a similar graphical representation to FIG. 3, extended to cover a wider time period of the participant's psychedelic journey, from preparation to integration. FIG. 4 depicts anchoring VRPP (VR 2) and grounding VRPP (VR 0) across the various different sessions and, in a similar way to FIG. 2, represents application of the anchoring VR (PS-VR) in subsequent sessions (in this case, a debriefing session and a later integration session).

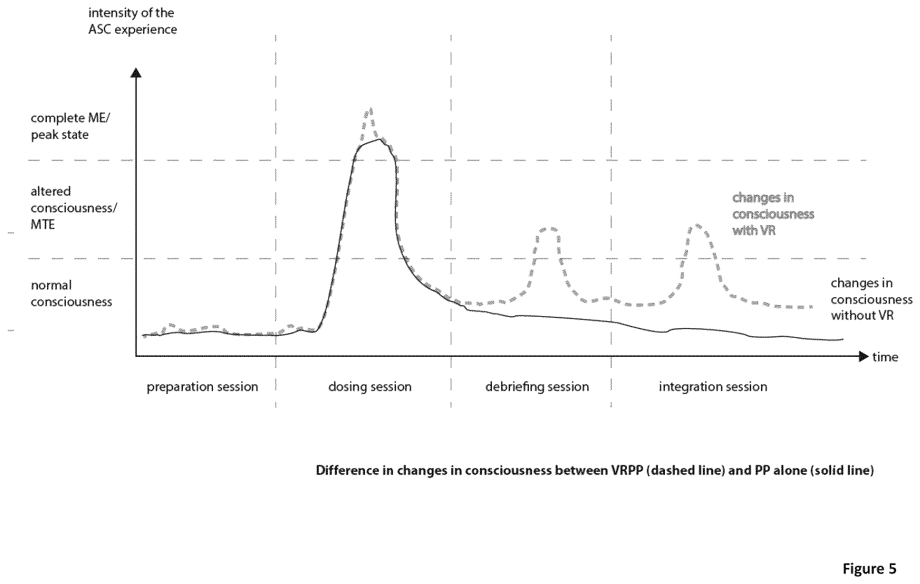

FIG. 5 provides a similar graphical representation to FIG. 4, but illustrates a comparison between the VRPP of the invention and PP alone. The raised peak in the dosing session represents the enhancement to the peak experience (which may equate to, for example, attainment of MTE) provided by the application of VR2 during the peak experience in the dosing session, while the new peaks during the subsequent integration sessions represent the effect of the peak anchor approach, ie. the evoking of peak sensations, emotions and mind states during these later non-dosed sessions.

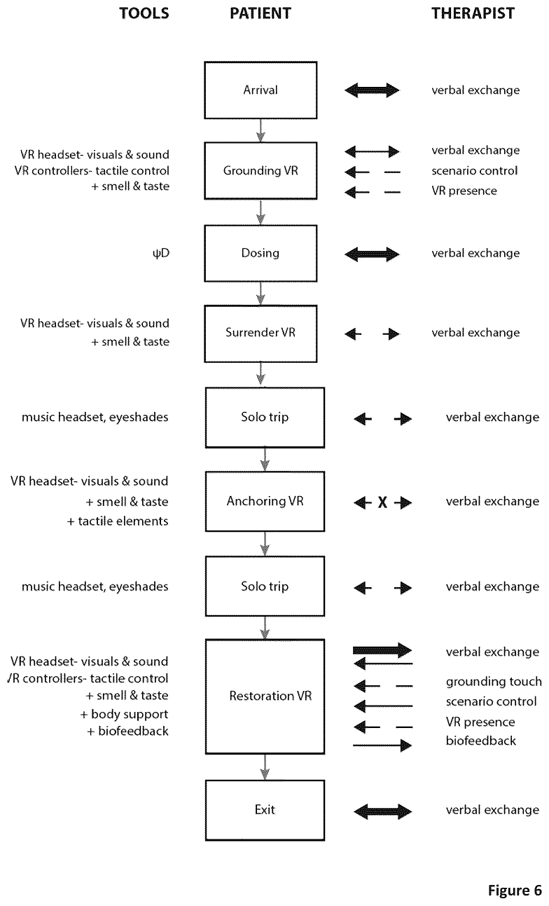

FIG. 6 depicts in a rather different form to FIG. 3 the timeline and elements of an example dosing session, including information regarding the therapeutic interactions (between therapist and participant) and the various tools used.

### Annex 1—Protocols. Treatment Approach, Indications and Contraindications

1.0 Introduction

As will be understood, these protocols are intended for use only by qualified therapists and only with participants who have provided their informed consent. While the invention is rooted in traditional principles of the ‘set and setting’ approach for psychedelic-assisted psychotherapy (PAP) or psychedelic therapy (PT), it introduces a novel and beneficial application of VR in many or all of the phases of treatment. It can also be extended to the use of VR in the additional phase of self-practice. As explained further below, the VR scenarios are applied to the participant to provide a continuous, multisensory stimulation that facilitates movement through treatment, augments therapy outcomes, and fosters psychological support.

1.1 Treatment Approach

VR is used in combination with a therapeutic dose of psychedelic compound which contains psilocybin. Therapeutic dose are between 0.1 mg-0.6 mg per 1 kg of body weight (Moreno et al. 2006; Brown et al. 2017), or between 10 and 50 mg of psilocybin total (Johnson et al. 2014; Carhart-Harris et al. 2016).

Sections below outline VR mechanisms that are uniquely suited to PP. Further sections will expand on these by i) outlining specific elements to achieve these functions, ii) explaining how to use each mechanism in every phase of treatment iii) propose VR scenarios that make use of those mechanisms in ways that are compatible with PP features. 1.2 Elements of the Therapy Method
Participant safety and wellbeing are always prioritized.
A qualified therapist with the appropriate training and experience relevant to the methods in this manual is required.
The therapy requires a thorough understanding of the nature of psychedelic effects and the non-linear manner in which they can lead to healing effects.
Providing adequate participant preparation and orientation to both elements of the therapy, PP and VR, is vital.
The development of therapeutic alliance/rapport and trust over the course of therapy is essential.
Creating an appropriate set, setting, and support system during all phases of treatment is essential.
VR is introduced in one of first preparation sessions to increase comfort and familiarity with the VR headset and general experience of VR in anticipation of use throughout the rest of therapeutic sessions
Although psychedelic-assisted psychotherapy is divided into three phases: preparation, dosing and integration, a maximum continuity should be ensured throughout all sessions. This is achieved with either one of VRP protocols, as well as any additional techniques if applied (e.g. breathwork, nurturing touch, etc.)
A nondirective approach to therapy based on empathetic rapport and empathetic presence should be used to support the participant's own unfolding experience and the body's own healing process. A non-directive approach emphasizes invitation rather than direction.
Grounding VR provides a continuous, multisensory catalyst throughout the entire psychedelic experience, is nondirective, self-selected, with low intensity stimuli and wide focal points of minimal attention. It favors relaxation, buffering, surrender and therapeutic alliance mechanisms, and is used during each phase of treatment.
Additional VR features can be added in to support specific functions or mechanisms that preparation or integration therapy wants to address, for example to support core ACT principles of Cognitive Defusion, Acceptance, Awareness, Transcendent Self, Values and Action. These are used during appropriate sessions and can be included or opted out on one-on-one basis, as the therapist sees fit.
Anchoring VR is used when peak effects are reached during dosing to anchor mystical or peak experience with a short, targeted application of a multisensory stimulation that is novel. It should be applied when emotions reach the highest intensity.
Facilitation VR is used prior to dosing, to prepare and train the client in achieving non-ordinary states of consciousness, and during dosing, to guide transition into and out of non-ordinary states
It is essential to encourage the participant to trust her/his inner healing intelligence, which is a person's innate capacity to heal. it is important to highlight the fact that the participant is the source of her/his own healing. In many ways, the therapy and the therapists can be seen more as facilitors, facilitating access to (rather than being the source of) the healing process.
During dosing, VR supports emotional experience while avoiding distraction from participant's inner healing journey. It is applied at different points during the dosing session and should always be removed at request of the participant or when he/she wants to turn completely inwards and focus on internal processing.
Therapists must attend to balancing their responsibilities as facilitators and as noninvasive empathic witnesses.
Intervention in the form of guidance or redirection, when deemed appropriate, can be used to facilitate the participant's processing.
Intervention in the form of adjusting the VR experience is appropriate at the request of the client, when deemed restrictive or non-conductive of the experience, or when deemed appropriate for other reasons.
The therapy should enable the processing of challenging experiences rather than their avoidance; however, this should be done with respect for protective mechanisms, which are referred to in different models of therapy as “resistance”, “defenses”, “protectors” etc. The therapists should facilitate awareness of and curiosity about any apparent resistance that arises rather than simply attempting to overcome it.
Therapists and VR are there to maximize the benefits of the inner experience catalyzed by psychedelics, while at the same time ensuring that the participant is safe and any potential psychological side effects, most prominently anxiety, are mitigated or resolved.
VR scenarios should assist in addressing distress. When needed, additional therapeutic techniques may also be used to that end, including nurturing touch, focused bodywork, breathing techniques.
Integration is viewed as an essential component of psychedelic journey and the effects of mystical or peak experiences should be taken advantage of during integration sessions.
VR scenarios are used in follow up integration sessions to evoke mystical or peak experiences that were achieved with dosing. By providing the same, targeted, multisensory stimulation that was applied during the intense emotional state, it eases the recall of non-ordinary states of consciousness so they can be integrated into daily life.
During integration, with the help of VR and psychotherapeutic approach the therapists aims to explore positive and negative emotional states evoked with psychedelics, address any difficulties, insights or queries that emerged as the result of the psychedelic session
Patients should be given time to first enter the emotional state with the use of VR before any verbal exchange takes place. Ineffable recall should be supported.
Exchange with a therapist can take place when the client is in the VR (VR-face exchange), after the VR session (face-face exchange) or with the use of a therapist avatar (VR-VR exchange). This should be tested during preparation and decided on by the client.
The participant is trained how to use VR and utilize VR scenarios on their own, in ways which have to be convenient to use with tools that are easily available to him/her outside of therapeutic space (for example, on a laptop at home). Follow-ups are organized to ensure that the VR scenarios remain usable and useful after integration sessions are finished or discontinued.

2.0 Indications and Contraindications

Indications: VR is indicated for any legal, intended use of therapeutic dose of psilocybin. this protocol is intended for use by anyone who guides, facilitates, sits, provides psilocybin with the intention to use it as treatment, therapy; for any mental health condition that psychedelics have been indicated for Contraindications: based mostly on psilocybin (can we state—‘all contraindications of psychedelic use’ or need to detail them here too? VR contraindications: serious problems with eyesight/peripheral vision; strong seasickness; epilepsy; perhaps psychosis/schizophrenia; younger than 12 y/o (irrelevant with psychedelics)

3.0 VR-Psllocybin Protocols

VR provides a safe, comfortable environment, which the user has control over, creating a favorable setting that is of importance to each therapeutic session. VR scenarios used in each phase of treatment unify them, ensuring continuity across all sessions, and facilitating movement through all phases of treatment.

Three main VR-Psilocybin Protocols (VRPP), that utilize VR as the moderator for psychedelic therapy, and their key functional mechanisms, are distinguished

Grounding VRPP
Relaxation
Mindful Presence
Buffering

Anchoring VRPP
Anchoring
Awe
Afterglow

Facilitation VRPP
ASC Priming
Surrender
Restoration

Special case (applied in emergency)
e Rescue

3.1 Grounding VRPP

The key role of the VR scenarios referred to as grounding is to maintain continuity across the entire psychedelic therapy, by providing a relaxing, safe and comfortable setting that unifies all sessions that revolve around psychedelic use while detaching the engagement with external distractions or everyday affairs.

Grounding VR scenarios utilize VR's unique ability to act as a buffering tool and to promote the state of relaxation and mindful presence. Buffering provides isolation from the external world, detachment from familiar temporal and spatial reference points, and anticipates deep immersion into the alternative world of sensory experiences. Mindful presence promotes a sense of arrival and presence at the space, smoothing the transition from daily life to therapeutic space. Relaxation relieves pre-session anxiety, enables a sense of ease, familiarity and control. Through a repeated use, Grounding VR scenarios trigger the sense of detachment, relaxation and presence that becomes more pronounced and easier with each session. The familiarity offers a sense of comfort and security; while it's pronounced difference to familiarity and comfort of other, daily cues, give the point of difference from daily life.

Grounding VR scenarios can be applied for between 5 min and 1 hr, once or several times during the session, and are used predominantly at the beginning of each session: during psychotherapy meetings that comprise both the preparation and integration phases, and during the preparation for psychedelic experience that immediately precedes dosing. The more consistency is kept across sessions, the better the effect: ideally, the exact same or very similar scenario is used each time, at the same time in each session (for example, a couple minutes after the session begins) and for the same duration.

Grounding VR scenarios can be fully interactive with audio, visual and haptic components; minimally interactive; or non-interactive (display only). The therapist may be present in the form of an avatar, visual or vocal representation or a recording. Other members of the group, if group therapy is involved, can be present as avatars, visual or vocal representations, or recordings.

Grounding VRPP

Preparation Phase
1. Arrival
2. Introduction talk with the therapist
3. Getting comfortable, sitting down standing
4. Grounding VR scenario on, possible range 5 min-1 hr
5. VR off
   a. Repeat 4-5 if needed
6. Therapeutic engagement, for example talk therapy (non-VR)
7. End of session
   Dosing phase
1. Arrival
2. Introduction talk with the therapist
3. Getting comfortable, sitting down standing
4. Grounding VR on, possible range 5 min-1 hr
5. VR off
   a. Repeat 4-5 if needed
6. Preparation for dosing session—check up, toilet visit, set up of space, food and music
7. Getting comfortable, sitting down/standing/lying down
8. Dosing
9. Getting comfortable, sitting down/lying down
10. Putting blindfolds and music on if needed
11. Solo trip with trp sitter
   a. If needed: Rescue VR on, possible range 5 min-1 hr, dependant on participant's emotional state
   b. VR off
   c. Solo trip with sitter continued
   d. Repeat a-c if needed
12. Short debrief following the trip
13. End of session
   Integration phase (including debrief sessions)
1. Arrival
2. Introduction talk with the therapist
3. Getting comfortable, sitting down/standing
4. Grounding VR scenario on, possible range 5 min-1 hr
5. VR off
   a. Repeat 4-5 if needed
6. Therapeutic engagement, for example talk therapy (non-VR)
7. End of session

3.1. Relaxation

VR interventions reliably facilitate an immediate state of relaxation which is useful for all phases of psychedelic treatment.

Anxiety is the most common psychological adverse effect of psychedelic use, both in recreational and therapeutic settings (USONA, 2018). Pre-session relaxation improves therapy outcomes. Client's sense of presence and perception of therapy's effectiveness are positively affected when relaxation is used prior to counselling (Dunn et al. 2013). A relaxed, open mind-set prior to and during a psychedelic experience is key to therapy's success (USONA, 2018). A relaxed setting during the dosing session also has a major impact on the psychedelic experience. Successful relaxation techniques significantly contribute to benefits following the treatment of all affective disorders, including PTSD, OCD and addiction. Observed benefits include reductions in anxiety and depression scores (Manzoni et al. 2008; León-Pizarro et al. 2007), improved physiological markers of stress response (Esch et al. 2003) and self-reported improvements on physical discomfort (León-Pizarro et al. 2007).

VR relaxation sessions can significantly decrease physiological markers and self-reports of state-anxiety, for example when a 7 minute breathing exercise is used among children (Van Roiji; 2016). PET scans showed improved limbic-regulated response of reward in alcohol dependent patients when VR-based relaxation was applied prior to exposure treatment (Son et al. 2015). In an acute stress relief intervention, VR group showed substantial improvements in stress response, which were consistent across self-reports and physiological measures of heart rate and cortisol levels, and significantly different from other stress-relief interventions (Uszio, 2018). VR has proven particularly effective for patients who were uncomfortable with all other forms of treatment (Maples-Keller, 2017).

3.1.2. Mindful Presence

Being distracted or disengaged is one of the key challenges in therapy. Having to face difficult or painful memories, address challenging emotions of guilt, disappointment or anxiety, or the challenge of opening up can lead to a flight response, provoking patients to discontinue therapy or attend it without fully engaging in it. While novelty and intensity of the psychedelic experience is very successful at attracting attention, psilocybin does allow for a degree of mind wandering or distracting oneself, for example by chatting to the therapist, if a participant is resistant to the effects (Bourzat & Hunter, 2019). This can be mitigated by a sense of presence and commitment to staying present.

Psilocybin combined with meditation facilitates positive changes in psycho-social functioning, which are thought to be associated with subjective ego-dissolution experiences (Smigielski et al. 2018). Contemplative mind-set combined with psilocybin-occasioned mystical experiences determines lasting improvements on prosocial attitudes and healthy functioning (Griffiths et al. 2018).

VR has proven highly successful in helping people engage with mindfulness, reach a meditative state (Perhakaran et al. 2016; Seabrook et al. 2020) and enjoy more significant mental health benefits of that state as compared with controls (Tarrant et al. 2018). State mindfulness and positive affect significantly increase after VR-enhanced mindfulness (Seabrook et al. 2020). VR-enhanced meditation led to EEG patterns unique to relaxed, meditative state, unobserved in controls (shift of high to low Beta frequencies, reduced Beta activity in anterior cingulate cortex; Tarrant et al. 2018).

3.1.3. Buffering

While current psychedelic therapies acknowledge the importance of stimulus and environmental setting in psychedelic experience, isolated visual (Johnson et al 2014) or auditory (music) cues they employ are stripped off their important role. Much like many other altered state methods, psychedelic therapy encourages insulating oneself from the external processes, therefore sensory cues should not only offer positive stimulation but, even more importantly, buffer negative or distracting stimulation.

The loss of the sense of space and time during the psychedelic experience engages patients in the process of contemplation and introspection (USONA, 2018). Absorption into a VR scenario can elicit such effects. The loss of sense of space, time and even connection to one's body is highly common. Patients tend to lose points of reference with reality, which engages them in the process of contemplation and introspection (Seabrook et al. 2020). Engagement with VR scenarios can be stronger than with the real world, cushioning its impact on current psychological and emotional state (Kotler & Wheal, 2017).

VR can be used to replace familiar settings, full of cues and symbols which may distract the mind and maintain engagement in the thought processes of the day to day, with cues and symbols that are most conductive of a deep immersion into the journey. Introduction of colors, sounds, landscape, use of art have a substantial, significant impact on the healing process during therapy (Iyendo et al. 2016) and VR is an easy, effective tool to facilitate all that. Finally, VR gives a sense of personal control over a therapeutic environment, which enhances therapeutic outcomes (Huisman et al. 2012).

3.2 Anchoring VRPP

Scenarios referred to as anchoring use synergistic properties of VR and psychedelics to augment the emotional experience during the peak state, and to create anchors of peak experience that can be used after the psychedelics effects wane, to trigger their recall, ease access to those otherwise ineffable experiences, and therefore maximize therapeutic use of experiential moments during integration.

VR has the capacity to offer stimuli that are unprecedented and unexpected. Applied during the peak experience, this type of stimuli demands attention, renewing focus, awareness or sense of agency over the experience; injects new vigor and enthusiasm in order to continue further exploration or dive deeper into it; deepens the sense of surrender to the journey; and relieves any fatigue, sleepiness or tiredness sometimes caused by psychedelics.

Synergistic use of this multisensory, highly absorbing VR stimulus and peak psychedelic effects capacitates them to augment emotions and sensations in unparalleled ways, producing states of awe. VR augments perceptual, psychological and emotional sensations brought on by psychedelics, deepening the ability to engage with states of gratitude, connectedness, self-realization, mindfulness, love or forgiveness; as well as surrender to challenging emotions including pain, vulnerability, inadequacy, irrelevance or loneliness. Psychedelics increase absorption into the VR world, and hence the sense of immersion and relatedness to VR scenarios that produce amazement, aesthetic arrest, joy, admiration or wonderment.

Through jointly producing such profound, unprecedented states, VR and psychedelic experiences become linked. A targeted, multisensory VR stimulus becomes an anchor for those peak sensations, emotions and mind states, attributing it with the capacity to evoke them during non-dosed sessions. Peak anchors are achieved with scenarios that use specific, unfamiliar, non real-world elements, that are conducive to emotional release.

Following the dosing session, VR anchors play a role in facilitating integration of psychedelic experiences. Anchoring improves access to experiences that are impossible or difficult to communicate while under the influence of the drug. One of the key advantages of capitalizing on the effects of awe and peak anchors is the expansion of afterglow. Repeated use of peak anchors that hold strong emotional links to peak experience, maintain the unique psychological and emotional flexibility, openness and positive mood that are characteristic of afterglow, extending the time available for therapeutic engagement when it has the highest potential to bring beneficial outcomes.

Anchoring VR scenarios are introduced during the peak phase of the dosing session, which occurs differently for every individual, most commonly at 1-3 hrs post dosing. It can be applied for between 5 and 30 mins, once or several times within that period. The exact same anchoring VR scenario is then used during the integration phase—both in the initial debrief sessions and during further integration sessions. To maximize its potential, the first few debrief sessions that use anchoring VR scenarios should be shortly after the dosing session, as often as feasible in the first 2-3 weeks (for example, every other day). Anchoring VR scenarios are used in the same way during the rest of the integration and, if possible, provided as a VR, 3D or 2D scenario to be used by the participant at home for as long as they want after the integration process ends.

Anchoring VR scenarios should be minimally interactive with audio, visual and haptic components; or non-interactive (display only).

Anchoring VRPP

Dosing Phase
1. Arrival
2. Introduction talk with the therapist
3. Getting comfortable, sitting down standing
4. Preparation for dosing session—check up, toilet visit, set up of space, food and music
5. Getting comfortable, sitting down/standing/lying down
6. Dosing
7. Getting comfortable, sitting down/lying down
8. Putting blindfolds and music on if needed
9. Solo trip with trp sitter
   a. If needed: Rescue VR on, possible range 5 min-1 hr, dependant on participant's emotional state
   b. VR off
   c. Solo trip with sitter continued
   d. Repeat a-c if needed
10. Anchoring VR on, at between 1 hr and 3 hrs; possible range 5 min-30 min
11. VR off
   a. Repeat 10-11 if needed
12. Solo trip with trp sitter continued
   a. If needed: Rescue VR on, possible range 5 min-1 hr, dependant on participant's emotional state
   b. VR off
   c. Solo trip with sitter continued
   d. Repeat a-c if needed
13. Short debrief following the trip
14. End of session

Integration Phase (Including Debrief Sessions)
1. Arrival
2. Introduction talk with the therapist
3. Getting comfortable, sitting down/standing
4. Anchoring VR scenario on, possible range 5 min-30 min 5. VR off
6. Therapeutic engagement, for example talk therapy (non-VR)
7. End of session

3.2.1. Awe

Awe is an effective medium for increasing positive emotions (Piff et al. 2015). Compared to other forms of mood boosters, including meditation, relaxation or therapy, it is instantaneous, has no prerequisites and the effect is irrelevant of predisposition to experience awe. Awe's key mechanism—the need to accommodate vastness—clears away chaotic thoughts and emotions. Even just a brief experience of awe can significantly improve mood, for example by relieving anxiety associated with uncertain life events (Rankin et al., 2019). A psychedelic experience is an example of such an uncertain event, which is packed with unexpected, sometimes challenging thoughts or sensations, that should be fully committed to, regardless of their emotional load. There are also parallels between the sensation of awe and ineffable, numinous, transcendental and mystical characteristics of psychedelic experiences. Experiencing awe leads to personal growth that is immediate and grand enough to compare it to quantum changes attributed to psychedelics (Hendricks, 2018). Existential feeling of awe may in fact moderate psychobehavioral changes that emerge following the psychedelic journey. Awe-related perception of vastness and the consequential need for accommodation, accompanied by restructuring of (Hendricks, 2018).

VR has proven to be highly successful in facilitating the experience of awe. VR is the ideal medium for evoking the challenging, destabilizing and deep sensations associated with awe, classifying it as a self-transcendental experience (Chirico et al. 2018). What is more, VR-induced awe has a functional relationship with psychedelic mechanisms. Recent findings on phenomenological aspects of awe distinguish between the ‘quick boiled awe’ and the ‘slow simmer awe’ (Chirico et al. 2018). VR-based experiential approach allows for blending of multi-faceted attributes of awe (the positive as well as the challenging ones), thus eliciting the latter, more intense form (Chirico et al, 2016/2017; Gallagher et al., 2015), which has been proposed as a putative mechanism of psychedelic effects (Hendricks, 2018).

Environmental settings that evoke awe impact the depth of an altered state and the mystical experience on psychedelics (Doblin, 1991). Lowered reality testing mechanisms and increased immersive tendencies caused by psychedelics augment the sensation of awe that is evoked by the awe-producing VR scenarios, which in turn guide the sensation of awe for the remaining part of the psychedelic trip.

3.2.2. Peak Anchors

The anchoring process utilizes a unique, multisensory set of cues, which by being applied during a highly potent emotional state become mentally attached to it. Anchors then enable evoking that same emotional state at will through application of those exact cues. The success of constructing anchors as well as their potency are highly dependent on the intensity of the source emotion. Psychedelic experiences are perfect candidates as the source of emotional state, because of their unusually high level of intensity as well as the strength of impact they exert on a wide range of psycho-emotional mechanisms. Having access to psychedelic-evoked emotions that are devoid of other effects of the drug, most notably the inability to communicate in a cohesive manner, increases chances of integrating those effects and gives therapists and clients more time to work through interesting, unexpected or challenging emotions that may arise in the process.

Despite the fact that ineffability is one of the key characteristics of the psychedelic experience, in current integration procedures recall is based largely on verbal exchange. If direct, verbal cues are applied too quickly, deeper, less explicit layers of the experience may be lost. Prompting the memory of peak experiences without direct verbal cues but with multisensory anchors, means that recalling those emotions will meet less resistance from the conscious mind and become more exploratory. VR anchors are a perfect medium to facilitate this ineffable internal recall. VR is an anchoring catalyst that is symbiotic with unique characteristics of the psychedelic state. VR-evoked experiences share unique properties of the psychedelic state. Immersion in the alternative, virtual reality also tends to be ineffable. Absorption into VR or into a psychedelic state often counteracts reality testing mechanisms, tricking the senses into making the surreal scenario appear real (Glowacki et al. 2020; Suzuki et al. 2017). Engagement with VR scenarios and engagement with psychedelic experiences can be stronger than with the real world, affecting perceived oneness with others and the self (Kotler & Wheal, 2017). The loss of sense of space, time and even connection to one's body is highly common with both practices (Seabrook et al. 2020).

VR is a multisensory medium, with properties that are synergistic to psychedelic states, that offers a novel, unique and intense stimuli that can be reapplied multiple times in a precisely the same manner. VR is an ideal tool to anchor the source emotional states that accompany the peak psychedelic experience and release it at will later, in order to aid the integration process, translating powerful but ineffable states into real world outcomes.

3.2.3. Afterglow

Afterglow, a short period of time when beneficial psychological and emotional effects of the psychedelic experience remain at a highest level, has been recognized as the most potent time for integration and achieving substantial therapeutic outcomes (Majic et al. 2015; Bowen et al., 1970; Pahnke et al., 1970). Its powerful nature is characterized by ‘elevated and energetic mood with a relative freedom from concerns of the past and from guilt and anxiety’ and the enhanced willingness ‘to enter into close interpersonal relationships’ (Pahnke, 1969). Some researchers suggest that better long-term effects achieved by frequent dosing, as opposed to a single dose, are due to maintenance of the afterglow effect, which is especially important during the time when the risk for relapse is the highest (Johnson et al. 2014). In several traditional rituals, the next dose is given only after the afterglow subsides, respecting the role that this phase plays in healing (Majic et al. 2015).

Enduring through challenges brought on by integration is also moderated by openness, plasticity and non-judgement sustained during the afterglow (Johnson et al. 2014). But the process of integration cannot be hurried and the afterglow wanes with time. Legal status, cost, and potential adverse effects of psychedelic compounds mean that more than 3 dosing sessions rarely occur in the clinical program, which is advised to last around 6 months, making it to one dose per 2 months. In contrast, the afterglow is known to last about 2 weeks. Structuring additional peak-evoking scenarios between dosing counteracts this disproportion.

Additionally, psychedelic effects seem to display a level of habituation and, after reaching the peak experience, repeated dosing does not seem to escalate the effect further. If the afterglow effect is maintained for longer, beneficial psychedelic effects can be maximized even without frequent dosing.

VR-based anchoring process is a replacement for repeat dosing, that can be used as many times as necessary and without any limitations that the repeat dosage entails. With the capacity to evoke emotional states that accompanied the psychedelic experience, VR maintains their effects for longer. Used repeatedly during the afterglow, VR-based peak anchors extend this beneficial time, aiding prevention of habituation to peak experiences and therefore waning of their effects with time. VR is a comfortable, easy to use method that is keenly adopted by patients during therapy. Further, as a digital tool, VR-based scenarios can be used to extend the integration phase without restrictions of duration, enabling self practice at home with the use of inexpensive VR glasses, computer-based 3D displays or 2D videos, that repeat the same, familiar cues for as long as it brings benefit or comfort to the client.

3.3 Facilitation VRPP (Surrender VRPP and Restoration VRPP)

VR scenarios, referred to as facilitation, offer a gentle guidance for the psychedelic experience through adequate preparation for the trip and provision of a favorable setting for the critical time of transition between daily consciousness and an altered state of consciousness (ASC).

ASC priming scenarios are used to prime occurrences of expanded states of consciousness, practice an ability to achieve an altered state of consciousness and promote the sense of ease around obtaining and experiencing those states, including perceptual alterations. They are also used to train methods for deep exploration of accompanying sensations and methods of communication of experiential moments in therapy, which may be produced by altered states, in particular ineffable experiences. Surrender scenarios offer stimulus capable of disrupting the participants' usual internal dialogue, to encourage a stronger level of physical, psychological or emotional engagement and to encourage freedom to dwell in unfamiliar psychological and emotional states and thus catalyse the occurrence of mystical and peak experiences They prepare the participant for what is to come, challenge the participant emotionally and psychologically to stay alert, pay attention and maintain a high level of awareness, encourage focus rather than avoidance, and nudge to give in to the impending experience. Restoration scenarios are used to ease the transition back from an altered state of consciousness, offer a buffer time between an ASC and normal consciousness, cushioning the clash between the two realities, offer comfort and support for the return to wakeful consciousness. Restoration time facilitates the in-between state where experiences brought on by the peak phase continue to be vivid while the mind slowly reestablishes its ability to connect and communicate with the external world, shepherding the emergence of the ‘first narrative’—an early account of the first impressions of the trip, which is often disjointed and chaotic but most intimately resembling the actual emotional state of the participant.

Facilitation VR scenarios differ depending on their function. ASC priming scenarios are used during preparation sessions for between 5 min and 1 hr, once or several times during each session. Surrender scenarios are used at early stages of the dosing session, immediately or shortly after dosing between 0-60 min post dosing, during the pre-peak phase, for between 5 min and 45 min. Restoration scenarios are used during the dosing session, after the peak effects begin to wane between 4-6 hrs post dosing, during the post-peak phase, for between 5 min and 1 hr. Ideally, though the functions are distinct and require diverse mechanisms of action, facilitation VR scenarios are unified through their design, aesthetics and sensory cues into an ongoing journey.

Facilitation VR scenarios should be minimally interactive with audio, visual and haptic components; or non-interactive (display only). The therapist may be present in the form of an avatar, visual or vocal representation or a recording. Other members of the group, if group therapy is involved, can be present as avatars, visual or vocal representations, or recordings.

Facilitation VRPP (Surrender VRPP and Restoration VRPP)

Preparation Phase
1. Arrival
2. Introduction talk with the therapist
3. Getting comfortable, sitting down/standing
4. Facilitation VR scenario on (ASC Priming), possible range 5 min-1 hr
5. VR of
   a. Repeat 4-5 if needed
6. Therapeutic engagement, for example talk therapy (non-VR)
7. End of session

Dosing Phase
1. Arrival
2. Introduction talk with the therapist
3. Getting comfortable, sitting down/standing
4. Preparation for dosing session—check up, toilet visit, set up of space, food and music
5. Getting comfortable, sitting down/standing/lying down
6. Dosing
7. Getting comfortable, sitting down/lying down
8. Facilitation VR scenario on (Surrender), at 0 min-60 min post dosing, possible range 5 min-45 min
9. VR off
10. Putting blindfolds and music on if needed
11. Solo trip with trp sitter
   a. If needed: Rescue VR on, possible range 5 min-1 hr, dependant on participant's emotional state
   b. VR off
   c. Solo trip with sitter continued
   d. Repeat a-c if needed
12. Facilitation VR scenario on (Restoration), at 4-6 hr post dosing, possible range 5 min-1 hr
13. VR off
14. Short debrief following the trip
15. End of session

3.3.1. ASC Priming

The strength of the mystical experience is one of the key predictions of positive outcomes of the psychedelic psychotherapy, including decrease of clinical symptoms and lasting changes in well-being (Haijen, 2018). A clear intention for, as well as previous experience with, mystical-type experiences are conducive of its occurrence and depth following an ingestion of the psychedelic compound. An ability to achieve an altered state of consciousness, which facilitates the occurrence of mystical and peak experiences, can be practiced. Studies on experienced meditators show that experience and skill practiced in achieving an ASC with meditation enhances the effects of psilocybin (Smigielski et al. 2019).

VR has been recognized as a tool capable of eliciting an altered state of consciousness already in the 90s (Glicksohn & Avnon, 1997). Since then, VR games have been used extensively in the entertainment industry to produce an entire range of ASC (Weinel, 2018). VR has also been highly successful in facilitating therapies that entail the production of altered states of consciousness, including meditation (Perhakaran at al. 2016; Seabrook et al. 2020). Most importantly, VR-based scenarios have been used to study psychedelic phenomenon and to elicit psychedelic-Ike mystical experiences. A hallucination machine induces visual alterations that closely resemble those brought on by classical psychedelics (Suzuki at al. 2017), while the Isness model successfully evokes mystical experiences that match those of highest therapeutic doses of the most potent psychedelic compounds (Glowacki et al. 2020). It has been suggested that use of VR as a prep tool for perceptual changes that occur during psychedelics may have beneficial effects on the expectation and pre-trip anxiety (Aday et al. 2020). By deepening the comfort and ability for entering and maintaining an altered state of consciousness, VR enables a more in-depth surrender and exploration of the psychedelic state, in result augmenting its therapeutic benefits.

3.3.2. Surrender

Psychedelics display a remarkable expectancy effect. Setting that encourages a mystical experience is more likely to evoke one than a retreat or laboratory. Administration of psilocybin among participants who attended religious service in a place of worship has been the source of the most meaningful life experience for 100% of followed up respondents (Doblin, 1991). In a more recent version of this ‘Good Friday Experiment’, where the procedure was closely replicated but in a clinical environment, a matched response was offered by only 67% of participants (Griffiths et al. 2006). Psychedelics can also trigger thoughts or sensations that are challenging, painful or problematic. These can last for a short time or precipitate the entire journey. Resisting the difficult experience obstructs therapy's beneficial effects, stopping the resolution of the issue and impeding the depth of the experience overall. Facing challenging experiences often translate to best therapeutic outcomes, and immersing in them instead of avoiding them seems to be the most effective way to disturb anxiety or panic that they may initially cause, and to make the most out of them during integration (Carbonaro, Bradstreet & Barrett, 2016).

VR is highly successful at creating immersive mystical, spiritual, awe-evoking or natural scenarios that are long recognized as most conductive settings for the psychedelic trip. Absorption in VR scenarios often counteracts reality testing mechanisms, tricking our senses into making the scenario appear real (Glowacki et al. 2020; Suzuki et al. 2017). When reality testing is impaired, perception needs to adapt to new reality, opening the mind to new possibilities (Glowacki et al. 2020). When familiar environmental cues are missing and the mind is in a state of relaxation, the process of letting go becomes more automatic. Entering this new, alternative reality leads to a sense of surrender (Seabrook et al. 2020). The familiar cause and effect cycle is broken, the new World is devoid of familiar rules. In such a way, participating in the VR creation primes the ability to relinquish any control, which is necessary for immersing oneself in psychedelic experiences, good or bad, beautiful or challenging, joyous or uncomfortable.

3.3.3. Restoration

Profound ASC experiences are difficult to assimilate into the familiar, daily life (Kotler & Wheal, 2017) and therefore pose a challenge for integration. The outstanding experiences that accompany the psychedelic state are often completely at odds with the familiar sensations characteristic of the normal, wakeful consciousness. While they last, communication, especially verbal one, is either unappealing or simply impossible. Unfortunately, the insight that appears obvious and profound while on drugs, is less clear after their effects wane. To increase access to immediate reflection on the psychedelic experience, therapists try to engage clients in the ‘first narrative telling’ as soon as the effects wane. This first narrative is phenomenological and avoids interpretation, welcoming chaotic and seemingly nonsensical expressions. Still, words often cannot embody the abstract intentions. Creative expression is often encouraged, with the use of paints or crayons or whatever is available and appropriate to establish a link to those ineffable states.

This transitions from psychedelic state to normal consciousness can be compared to the moment we wake up while we still remember what we dreamed of but awareness of the real world begins to creep in. As the type of an ‘in-between world’, VR extends the time spent in that state, where the psychedelic effects still echo, letting them solidify in the memory. While at this point the participant begins to be sober enough to already begin expressing oneself, it is still time when he/she is apprehensive about connecting with the real world or real life humans. VR allows for the first narrative to be formed and explored within the participant's own, abstract world, before it is shared externally. While holding an open dialogue around the psychedelic experience is the challenge for a therapist (Guss et al. 2019), most patients naturally gravitate towards VR as a more comforting and less confrontational tool than a face to face talk therapy (Garrett et al. 2020, Riva et al. 2019). Sharing of this intimate, sometimes illogical narrative feels less exposed in VR than during an eye to eye with a therapist. Finally, while use of other creative tools requires the return to the real world (opening the eyes, facing the light, seeing other humans, being back in the lab) VR gives an opportunity to use expressive tools while still immersed in this alternative universe that is continuous with the experience rather than putting an end to it. Creative expression in VR is less inhibited, activates full sensory engagement in the process of creation and makes it more playful and thus exploratory than 2D art (Kaimal et al. 2019). It also challenges our familiar perception, building a bridge between the expanded perceptual field of an ASC to the contracted perceptual field of daily consciousness.

3.4 Special Scenario: Rescue VRPP

Given that sitting through challenging experiences is the one of the most beneficial aspects of the psychedelic journey, therapists face a difficult challenge of distinguishing when they should encourage leaning into the challenge, the pain or otherwise difficult sensation, and when it becomes so overpowering that it is necessary to put on the breaks and offer comfort instead. At the moment, there is no tool out there capable of putting those breaks on immediately and reliably. The only thing therapists can currently offer in response to marked agitation is taking off blinds shades and doing breathing or other relaxation exercises. Even pharmacological interventions, like benzodiazepines, take time before they can be administered and take effect, and may not be accepted or tolerated by some clients. Having access to something that provides an immediate relief from overpowering emotions is a long recognized relief in itself that works for any psychological or emotional challenge and in any mental health condition.

An immersive, interactive, strongly engaging VR world offers a competing stimulus for the psychedelic to put on such immediate stop to an over-challenging or otherwise detrimental psychedelic experience. VR has the ability to disrupt the psychedelic journey, whenever it turns out to be too difficult, overly anxiety-provoking or no longer beneficial for the client. Immediate anxiety relief combined with strongly absorbing nature of VR environment work as a sobering tool. This function, referred to as rescue, although applied only when necessary, plays an important role of a safety net that is of particular importance during the dosing session. Rescue VR can be applied ad-hoc, as a stand-alone, even if no other VR scenarios have been used in other sessions. VR is safe, comfortable and intuitive enough to offer immediate relief, however, at least one introductory VR session is highly recommended pre-dosing, to establish familiarity with the use of VR.

Rescue VR can last between 5 min up to 1 hr and can be repeated as many times as necessary. It is advised for the rescue VR scenario to be either fully or minimally interactive, with audio, visual and haptic components. The therapist may be present in the form of an avatar, visual or vocal representation or a recording.

4.0 The Unified VR-Psllocybin Protocol

VRP-Protocols can be used separately or joined into a single, comprehensive protocol that is used in all phases of treatment.

Unified VRPP

Preparation phase
1. Arrival
2. Introduction talk with the therapist
3. Getting comfortable, sitting down/standing
4. Grounding VR scenario on, possible range 5 min-1 hr
5. VR off
6. Therapeutic engagement, for example talk therapy (non-VR) 7. Facilitation VR scenario on (ASC Priming), possible range 5 min-1 hr
8. VR off
9. Therapeutic engagement continued, for example talk therapy (non-VR)
10. End of session Dosing Phase
1. Arrival
2. Introduction talk with the therapist
3. Getting comfortable, sitting down/standing
4. Grounding VR on, possible range 5 min-1 hr
5. VR off
6. Preparation for dosing session—check up, toilet visit, set up of space, food and music
7. Getting comfortable, sitting down/standing/lying down
8. Dosing
9. Getting comfortable, sitting down/lying down
10. Facilitation VR scenario on (Surrender), at 0 min-60 min post dosing, possible range 5 min-45 min
11. VR off
12. Putting blindfolds and music on if needed
13. Solo trip with trp sitter
   a. If needed: Rescue VR on, possible range 5 min-1 hr, dependant on participant's emotional state
   b. VR off
   c. Solo trip with sitter continued
   d. Repeat a-c if needed
14. Anchoring VR on, at between 1 hr and 3 hrs; possible range 5 min-30 min
15. VR off
16. Solo trip with trp sitter continued
   a. If needed: Rescue VR on, possible range 5 min-1 hr, dependant on participant's emotional state
   b. VR off
   c. Solo trip with sitter continued
   d. Repeat a-c if needed
17. Facilitation VR scenario on (Restoration), at 4-6 hr post dosing, possible range 5 min-1 hr
18. VR off
19. Short debrief following the trip
20. End of session

Integration Phase (Including Debrief Sessions)
1. Arrival
2. Introduction talk with the therapist
3. Getting comfortable, sitting down/standing
4. Grounding VR scenario on, possible range 5 min-1 hr
5. VR off
6. Therapeutic engagement, for example talk therapy (non-VR)
7. Anchoring VR scenario on, possible range 5 min-30 min
8. VR off
9. Therapeutic engagement continued, for example talk therapy (non-VR)
10. End of session

During preparation, VR is used to establish an ideal mindset and offer continuity that will echo throughout treatment. During preparation VR is also used to prep clients for experiences associated with achieving an ASC.

During the beginning of each session, including preparation, pre-dosing phase of dosing session and integration sessions, VR is used to encourage state of relaxation, mindfulness and reflection, as well as the sense of connectedness with the world, nature, oneself and others.

During pre-peak of the dosing session VR is used to provide an ideal setting, buffer from external cues, distract from internal processes and guide an emotionally fueled and experientially focused journey.

During peak time of the dosing session VR is used synergistically with psychedelics to augment emotional and experiential moments of the journey, and to boost and re-energize the trip.

During the post-peak of the dosing session VR is used to ease the transition into normality, offer comfort and support for the return to wakeful consciousness and enable eliciting reflection on the ineffable psychedelic experience.

During overly challenging and unexpectedly threatening experiences that occur throughout the dosing phase, VR is used as a rescue mechanism which puts an immediate stop to the experience, calms down and restores normal functioning.

During integration sessions VR is used as a reminder of peak experiences and emotions that came up during dosing and as an enjoyable, easy, ineffable method of practicing communication, sharing and reliving those emotions, integrating lessons from dosing session into daily consciousness; Following integration VR is used to offer direction for self-paced, client-led integration and practice for an unlimited length of time.

Treatment Administration Example for Dosing Session
(including approximate times and plausible ranges for unified VRPP protocol with psilocybin)

| | Example | | | |
|---|---|---|---|---|
| | time | | | |
| | from | | Typical | |
| | dosing | | duration | |
| Step | (min) | Description | (min) | Plausible range (min) |
| 1 | | Arrival | | |
| 2 | | Introduction talk with the therapist | | |
| 3 | | Getting comfortable, sitting/standing | | |
| 4 | | Grounding VR | 15-30 | 5-60 |
| 5 | 0 | Psilocybin dosing | | |
| | | (capsule; psilocybin 25 mg) | | |
| 6 | 5-20 | Getting comfortable, sitting; | 5-20 | immediately post |
| | | | | dosing up to 30 min |
| 7 | 0-60 | Facilitation VR (surrender) | 20-35 | 5-60 (depending on |
| | | | | time of administration |
| | | | | of VR post dosing) |
| 8 | 45-60 | Getting comfortable, lying down | | |
| 9 | | Putting blindfolds and music on | | |
| 10 | 90/120 | Solo trip with trip sitter (VR off) | | 60-120 |
| 11 | | Identifying peak state (eg. via MEQ6) | | |
| 12 | | Taking blindfolds off | | |
| 13 | 90-180 | Anchoring VR | 5 | 1-30; possible multiple |
| | (once | | | applications |
| | peak | | | |
| | identified) | | | |
| 14 | 120 | Putting blindfolds and music on | | |
| 15 | | Solo trip with trip sitter continued (VR | | |
| | | off) | | |
| 16 | | Identifying end of peak phase | | |
| | | (participant feedback) | | |
| 17 | 4 hours | Taking blindfolds off | | |
| 18 | 240-360 | Facilitation VR (restoration) | 20-60 | 20-60 |
| | (once | | | |
| | peak | | | |
| | phase | | | |
| | ended) | | | |
| 19 | | Sitting up | | |
| 20 | | Short chat | | |
| 21 | | End of session | | |
| 22* | anytime | Recovery VR (on identifying psycho- | 5-15 | 5-60 |
| | | emotional crisis via participant | | |
| | | feedback) *if needed | | |

Treatment Administration Example for Integration Session
(eg. 1-3 days post dosing)

| | Time | | | |
|---|---|---|---|---|
| | from | | Typical | Plausible |
| | arrival | | duration | range |
| Step | (min) | Description | (min) | (min) |
| 1 | 0 | Arrival | | |
| 2 | | Introduction talk with the therapist | | |
| 3 | | Getting comfortable, sitting/ | | |
| | | standing | | |
| 4 | 15 | Grounding VR | 15 | 5-60 |
| 5 | 30 | Integration (talk with therapist) | | |
| 6 | 45/60 | Anchoring VR | 5 | 5-30 |
| 7 | | Integration continued (talk with | | |
| | | therapist) | | |
| 8 | | End of session | | |

What is claimed is:

1. A method of treating a condition in a participant, comprising:
administering to the participant a therapeutically effective amount of a psychedelic drug (ΨD);
identifying when the participant experiences an altered state evoked by the ΨD;
providing a virtual reality (VR) experience to the participant while experiencing the altered state, whereby a response associated with the altered state is evoked in the participant upon provision of the VR experience when the participant is substantially under no influence of the ΨD.

2. The method of claim 1, wherein the VR experience is a dynamic scenario comprising multiple portions.

3. The method of claim 1, further comprising:
when the participant is under the influence of the ΨD, identifying a portion of the VR experience corresponding to the altered state; and
when the participant is substantially under no influence of the ΨD, providing said portion of the VR experience to the participant in order to evoke the response associated with the altered state.

4. The method of claim 1, wherein identifying when the participant experiences an altered state evoked by the ΨD comprises suitable psychological, physiological, behavioral or neurological measures, or a combination thereof.

5. The method of claim 1, wherein identifying when the participant experiences an altered state evoked by the ΨD is performed in real time or at a subsequent time, or by way of a combination of the two.

6. The method of claim 1, wherein said altered state is a peak state.

7. The method of claim 1, wherein the VR experience provided to the participant while experiencing the altered state, to be later provided to the participant when the participant is substantially under no influence of the ΨD, is the VR experience provided to the participant at a particular point in time following the participant's experience of a peak state.

8. The method of claim 1, wherein providing the VR experience to the participant comprises providing a VR headset to the participant.

9. The method of claim 8, wherein providing the VR experience to the participant further comprises providing one or more ancillary sensory simulation devices.

10. The method of claim 1, wherein providing the VR experience to the participant includes providing an acoustic environment, which is either pre-recorded or offered in real time.

11. The method of claim 10, wherein the acoustic environment includes a soundscape and/or music and/or vocal guidance.

12. The method of claim 11, wherein the soundscape is synchronized with the VR dynamic scenario.

13. The method of claim 12, further comprising providing audio headphones to the participant.

14. The method of claim 1, further comprising providing a different VR experience to the participant as a rescue VR if it is determined the experience has become detrimental, wherein the rescue VR is different to the portion of the VR experience corresponding to the altered state.

15. A method of treating a condition in a participant, comprising providing a virtual reality (VR) experience to the participant when substantially under no influence of a psychedelic drug (ΨD), in order to evoke a response associated with an altered state, said VR experience having been previously provided to the participant while experiencing the peak state under the effects of a ΨD administered to the participant.

16. The method of claim 15, wherein the VR experience previously provided to the participant comprises multiple portions.

17. The method of claim 15, wherein the method comprises providing to the participant a portion of the VR experience which has been identified as corresponding to the altered state.

18. A method of treating a condition in a participant using a unified virtual reality psychedelic protocol (VRPP) protocol involving multiple treatment phases, the unified VRPP protocol comprising a plurality of VRPP sub-protocols applied during one or more of the treatment phases, the sub-protocols selected from the group of altered state of consciousness (ASC) priming, facilitation, grounding, surrender, restoration, anchoring and rescue wherein:
at least one of the treatment phases comprises administering to the participant a therapeutically effective amount of a psychedelic drug (ΨD); and
wherein all of the VRPP sub-protocols are unified by providing to the participant under each sub-protocol a VR experience including at least one portion or experiential element common to all the sub-protocols,
so to moderate functions within each treatment phase in a predictable and repeatable way.

19. The method of claim 18, wherein the multiple treatment phases comprise, in addition to one or more dosing sessions in which the therapeutically effective amount of a ΨD is administered to the participant, one or more preparation sessions and one or more integration sessions.

20. The method of claim 18, wherein the multiple treatment phases comprise one or more self-practice sessions.

21. The method of 18, wherein the method comprises selecting a participant having a condition identified as being potentially treatable by way of the method.

22. The method of claim 21, wherein the condition treated by the method of the invention is a psychiatric condition or another type of personal condition to which the participant is subject.

23. A method of facilitating the operation of a clinical or therapeutic service or practitioner, comprising providing guide protocols to the clinical or therapeutic service or practitioner for carrying out the method of claim 1.

24. A method of facilitating the operation of a clinical or therapeutic service or practitioner, comprising providing guide protocols to the clinical or therapeutic service or practitioner for carrying out the method of claim 15.

25. A method of facilitating the operation of a clinical or therapeutic service or practitioner, comprising providing guide protocols to the clinical or therapeutic service or practitioner for carrying out the method of claim 18.
